(19) United States
(12) Patent Application Publication
(10) Pub. No.: US 2020/0194102 A1
(43) Pub. Date: Jun. 18, 2020

(54) MODEL FOR HIGH-THROUGHPUT SCREENING OF ENDOCRINE DISRUPTOR AND METHOD FOR SCREENING SAME

(71) Applicant: Nanjing University, Nanjing (CN)

(72) Inventors: Wei Shi, Nanjing (CN); Haoyue Tan, Nanjing (CN); Qinchang Chen, Nanjing (CN); Hongxia Yu, Nanjing (CN)

(73) Assignee: Nanjing University

(21) Appl. No.: 16/714,745

(22) Filed: Dec. 15, 2019

(30) Foreign Application Priority Data
Dec. 14, 2018 (CN) 201811529809.8

(51) Int. Cl. G16C 20/40; G16C 60/00; G16C 20/30; G16C 20/70; C07K 14/705; G06F 17/18; G06N 5/02

(52) U.S. Cl. CPC G16C 20/40; G16C 60/00; G16C 20/30; G06N 5/025; C07K 14/70571; G06F 17/18; G16C 20/70

(57) ABSTRACT

The present invention provides a model for high-throughput screening of endocrine disruptors and a method for screening the same. In the present invention, primary structural alerts, secondary structural alerts and tertiary structural alerts of compounds are extracted according to a nuclear receptor, and then the primary structural alerts, the secondary structural alerts and the tertiary structural alerts form a nuclear receptor high-throughput screening model; hierarchical structural alert matching is carried out on target compounds through the nuclear receptor high-throughput screening model, and ligand-receptor binding mode analysis and semi-quantitative prediction of binding activity and disrupting activity are performed. According to the present invention, the defect in prior art that potential nuclear receptor-mediated endocrine disruptors cannot be effectively screened in high throughput is overcome, high-throughput screening of potential nuclear receptor-mediated endocrine disruptors can be performed, and receptor competitive activity and A-Anta activity of the nuclear receptor-mediated endocrine disruptors can be determined.

### TECHNICAL FIELD

The present invention relates to the field of virtual screening and activity prediction of endocrine disruptors, and in particular to a model for high-throughput screening of endocrine disruptors and a method for screening the same.

### BACKGROUND

The human body contains 48 nuclear receptors, 12 of which are one of the most successful molecular targets in the history of drug discovery. Each of the nuclear receptors has one or more homologous synthetic ligands used as drugs. Such nuclear receptors are called classical nuclear receptors. The classical nuclear receptors include androgen receptor (AR), estrogen receptor α/β (ERα/β), glucocorticoid receptor (GR), mineralocorticoid receptor (MR), progesterone receptor (PR), retinoic acid receptor α/β/γ (RARα/β/γ), thyroid hormone receptor α/β (TRα/β) and vitamin D receptor (VDR). The classical nuclear receptors are a kind of transcription factors regulated by natural hormones. The natural hormones cause a series of key events through the competitive binding of ligand-receptor, and finally regulate an endocrine system. However, in a large number of studies, it is found that some artificially synthesized and natural compounds can mimic or resist natural hormones and interfere with normal endocrine systems of human beings and wild animals. Such compounds are called Endocrine Disrupting Chemicals (EDCs). At present, many compounds, such as polybrominated diphenyl ethers, bisphenol A and pyrethroid pesticides, have been detected to have obvious disrupting activity on classical nuclear receptors. These chemicals have also received extensive attention. In order to screen for potential EDCs, people have developed various effective in vivo and in vitro experimental methods, including competitive binding, reporter gene, yeast two-hybrid and fluorescence polarization and other in vitro tests, mouse uterus weight gain in vivo tests, and the like. However, on the one hand, it takes time and effort to adopt these test methods. The test methods are also quite high in cost. On the other hand, there are thousands of chemicals in the environment, which are difficult to screen for one by one.

For screening for EDCs, the prior art also provides some solutions, such as the invention titled VIRTUAL SCREENING METHOD OF HUMAN TRANSTHYRETIN (HTTR) DISRUPTORS (patent publication number: CN106407665A, publication date: Feb. 15, 2017). This solution discloses a method for virtual screening of hTTR disruptors, and belongs to the field of methods for screening environmental EDCs. In the virtual screening process, chemicals are first classified based on ten radical groups, then a quantitative structure-activity relationship model is used to predict the disrupting effect of each type of chemicals on human transthyretin, and then according to a predicted effect value, it is determined whether the chemicals have the ability to disrupt the hTTR and the disruptive strength. The process for screening the hTTR disclosed in this solution is simple and reasonable, and can be applied to virtual screening of potential hTTR disruptors in application domains and priority setting of the potential disruptors. However, the deficiencies of this solution are that although this solution has a high throughput, the application scope is narrow and the mechanism explanation cannot be effectively given.

There is also the invention titled METHOD FOR VIRTUAL SCREENING OF NUCLEAR RECEPTOR-MEDIATED ENDOCRINE DISRUPTOR BASED ON MOLECULAR DYNAMICS SIMULATION (patent publication number: CN103324861A, publication date: Sep. 25, 2013). This solution discloses a method for virtual screening of nuclear receptor-mediated endocrine disruptors based on molecular dynamics simulation, belonging to the field of virtual screening and activity prediction of environmental suspected endocrine disruptors. In this solution, the optimized small molecules are docked with receptor files obtained by experiments or homologous modeling to form a complex, and then molecular dynamics simulation is performed by using a GROMACS software package. The movement locus of the 12th helix of the receptor is analyzed, and the analysis of the root mean square deviation of the spatial position over time is used to identify pollutants with receptor activity. Within the specified time, if the curve tends to be stable, it can be considered that the corresponding helix is positioned at a determined position, thus having biological activity. Then, by examining the location of the positioning, it is determined whether the receptor is an agonist or an antagonist. However, the deficiencies of this solution lie in that although the interaction relation between the ligand and the receptor has been studied from the mechanism, it takes a long time and cannot provide effective high-throughput screening means in the face of more than 100 million chemicals with CAS numbers.

A structural alert derived from the structure-effect relationship is a structural fragment of an active compound that is related to a specific biological activity and has a mechanism principle. Associating the structural alerts with molecular initiation events can remove complexity of a large amount of biological information from the source and provide a closer link between structural features and harmful outcomes. In addition, the structural alerts can also provide an analysis of biochemical action mechanisms, demonstrating the feasibility of high-throughput research on various endocrine disruptors. The structural alerts have been used to focus on target research in pharmacology and drug safety, and remarkable results have been achieved, such as screening for potential hepatotoxic compounds and mitochondrial toxic compounds and the like.

Although the structural alerts have been used as a supplementary method to study endocrine disruptors, there is still a gap in identifying potential endocrine disruptors by high-throughput screening. Utilizing the structural alerts to study ligand-receptor binding can be understood as the first step of generation of disrupting activity. Therefore, the structural alerts can be used as a deviation-free method to cluster endocrine disruptors from different sources and to link the disrupting activity with chemical structural features, thus providing a possibility for high-throughput screening of potential endocrine disruptors. The results of literature retrieval show that no report has been found on the construction of a method for high-throughput screening of potential nuclear receptor-mediated endocrine disruptors by using a hierarchical structure alert method. Therefore, it is necessary to design and implement a method for screening of potential endocrine disruptors.

### SUMMARY

### 1. Technical Problems to be Solved

An objective of the present invention is to overcome the deficiency of the prior art that potential nuclear receptor-mediated endocrine disruptors cannot be effectively high throughput screened, and provide a model for high-throughput screening of endocrine disruptors and a method for screening the same, which can perform high-throughput screening of potential nuclear receptor-mediated endocrine disruptors, and can be used to determine the receptor competitive activity and agonistic-antagonistic activity of the nuclear receptor-mediated endocrine disruptors.

### 2. Technical Solution

In order to achieve the foregoing objective, the technical solution provided by the present invention is as follows:

A model for high-throughput screening of endocrine disruptors provided by the present invention, where primary structural alerts, secondary structural alerts and tertiary structural alerts of compounds are extracted according to a nuclear receptor first, and then a nuclear receptor high-throughput screening model is formed by the primary structural alerts, the secondary structural alerts and the tertiary structural alerts; wherein the compounds have three kinds of in vitro experimental data of a competitive binding experiment, a reporter gene experiment and a cytotoxicity experiment; the extraction of the primary structural alerts: based on a PubChem fingerprint molecular fingerprint database, the primary structural alerts of the compounds are extracted through substructure frequency analysis and substructure percentage analysis; the extraction of the secondary structural alerts: the secondary structural alerts are extracted from compounds meeting the primary structural alerts by using SARpy software; and the extraction of tertiary structural alerts: the tertiary structural alerts are extracted from compounds meeting both the primary structural alerts and the secondary structural alerts by using the SARpy software.

Preferably, the nuclear receptor is any one of AR, ERα, ERβ, GR, MR, PR, RARα, RARβ, RARγ, TRα, TRβ and VDR.

Preferably, the substructure frequency analysis is performed by using formula

$$Frequency of a fragment = \frac{( {N}_{fragment}^{A} \times {N}_{total} )}{( {N}_{fragment \_ total} \times {N}_{A} )} ,$$

the substructure percentage analysis is performed by using formula

$$Percentage of a fragment = \frac{{N}_{fragment}^{A}}{{N}_{A}} or \frac{{N}_{fragment}^{I}}{{N}_{I}} ,$$

wherein the above-mentioned Frequency of a fragment denotes substructure frequency; Percentage of a fragment denotes substructure percentage; N_{fragment}^{A} is the number of active compounds containing the structural fragment; Nₜₒₜₐₗ is the total number of compounds in a whole data set; N_{fragment_total} is the total number of compounds containing the structural fragment; N_{A} is the total number of active compounds in the data set; N_{fragment}^{I} is the total number of inactive compounds containing the structural fragment; and N_{I} is the total number of inactive compounds in the data set.

A method for high-throughput screening of endocrine disruptors provided by the present invention, which adopts the foregoing model for high-throughput screening of endocrine disruptors, comprises the following steps:

step 1: acquisition of structural data

selecting chemical structures of target compounds without relevant activity data from a public database, and denoting the chemical structures by SMILES numbers;

step 2: determination of a target nuclear receptor

determining a target nuclear receptor based on the disrupting activity of the target compounds to be predicted;

step 3: matching of primary structural alerts

selecting a target nuclear receptor high-throughput screening model based on the determined target nuclear receptor, calculating molecular fingerprints of the target compounds by using a PubChem fingerprints molecular fingerprint database of PaDEL-Descriptor software, and matching a calculated result with primary structural alerts of the target nuclear receptor high-throughput screening model;

step 4: matching of secondary structural alerts

grouping target compounds meeting the primary structural alerts based on the primary structural alerts, and matching target compounds with secondary structural alerts of the target nuclear receptor high-throughput screening model by using SARpy software;

step 5: matching of tertiary structural alerts

grouping target compounds meeting both the primary structural alerts and the secondary structural alerts based on the secondary structural alerts, and matching with tertiary structural alerts of the target nuclear receptor high-throughput screening model by using the SARpy software.

Preferably, a method for grouping the target compounds in step 4 comprises: grouping the primary structural alerts of the target nuclear receptor high-throughput screening model first, and taking the primary structural alerts containing polar atoms and aromatic ring bonds as Type 1; taking the primary structural alerts containing aromatic bonds as Type 2; taking the primary structural alerts of carbon chains containing oxygen atoms as Type 3; finally taking the primary structural alerts of any carbon chains without oxygen atoms and aromatic bonds as Type 4; wherein the priorities of Type 1, Type 2, Type 3 and Type 4 are successively decreased, matching the primary structural alerts of the target compounds with the primary structural alerts in each group, and then classifying the target compounds from the group with successful matching to the group with the highest priority.

Preferably, a method for grouping the target compounds in step 5 comprises: grouping the secondary structural alerts of the target nuclear receptor high-throughput screening model first, and taking the secondary structural alerts existing only in antagonistic compounds as Type 1-1, taking the secondary structural alerts existing only in agonistic compounds as Type 1-2, and finally taking the secondary structural alerts existing in agonistic-antagonistic (A-Anta) compounds as Type 1-3; wherein the priorities of Type 1-1, Type 1-2 and Type 1-3 are successively decreased, matching the secondary structural alerts of the target compounds with the secondary structural alerts in each group, and then classifying the target compounds from the group with successful matching to the group with the highest priority.

Preferably, the method further comprises step 6: analysis of ligand-receptor binding mode selecting target compounds meeting the primary structural alerts, the secondary structural alerts and the tertiary structural alerts through the target nuclear receptor high-throughput screening model, selecting a pattern protein receptor crystal from RCSB PDB Database based on the disrupting activity of the target compounds, performing molecular docking by using Autodock vina software, and performing ligand-receptor binding mode analysis by using Ligplus software;

step 7: semi-quantitative prediction of binding activity and disrupting activity

where target compounds with the same structural alert have similar ligand-receptor binding mode and similar binding activity; when the binding activity is positively correlated with the disrupting activity, grouping the target compounds based on the primary structural alerts, the secondary structural alerts and the tertiary structural alerts of the target compounds, and semi-quantitatively predicting the binding activity and the disrupting activity of the target compounds.

Preferably, the binding activity and the disrupting activity are standardized and classified in intensity by using formula

$$Activity value = - {log}_{10} \frac{{K}_{i} , {K}_{d} , {AC}_{50} , {IC}_{50} , {EC}_{50} ( uM )}{1000000} \text{:}$$

wherein Activity value denotes a value of activity intensity, Kᵢ denotes an inhibition constant, K_{d} denotes a dissociation constant, AC₅₀ denotes a median activity concentration, IC₅₀ denotes a median inhibitory concentration, EC₅₀ denotes a median effect concentration, uM denotes a micromolar weight, Activity value ≥7 denotes a strong activity intensity, 7>Activity value ≥6 denotes a medium activity intensity, and Activity value <6 denotes a weak activity intensity.

Preferably, the target nuclear receptor high-throughput screening model is any one of an AR high-throughput screening model, an ERα high-throughput screening model and a GR high-throughput screening model.

Preferably, the target compounds are organic compounds.

### 3. Beneficial Effects

Compared with the conventional prior art, the technical solution provided by the present invention has the following significant effects:

(1) According to the model for high-throughput screening of endocrine disruptors and the method for screening the same provided by the present invention, by constructing the model for high-throughput screening of endocrine disruptors and then matching the hierarchical structural alerts of the target compounds, the receptor competitive activity and A-Anta activity of the endocrine disruptors can be qualitatively determined; the structural alerts corresponding to different types of endocrine disruptors are identified by using three endocrine disruption mechanisms of agonist, A-Anta and antagonist, the relationship between the activity and the internal mechanism is successfully established, and the binding activity of the disruptors and the disrupting activity of some of the disruptors are semi-quantitatively predicted.

(2) According to the model for high-throughput screening of endocrine disruptors and the method for screening the same provided by the present invention, the novel model for high-throughput screening of endocrine disruptors based on structural alerts is constructed by using a novel method of hierarchical structural alerts, and the binding activity and the disrupting activity of the disruptors are semi-quantitatively predicted. The prediction effect is better than that of a conventional QSAR model.

(3) According to the model for high-throughput screening of endocrine disruptors and the method for screening the same provided by the present invention, by analyzing the ligand-receptor binding modes of the target compounds, which amino acids the target compounds interact with to produce disrupting activity can be predicted, and then the generation mechanism of the disrupting activity can be predicted.

(4) According to the model for high-throughput screening of endocrine disruptors and the method for screening the same provided by the present invention, compounds with the same structural alert have similar ligand-receptor binding modes and similar binding activity; when the binding activity is positively correlated with the disrupting activity, the compounds are grouped based on the primary structural alerts, the secondary structural alerts and the tertiary structural alerts of the compounds, the binding activity and the disrupting activity of the compounds are semi-quantitatively predicted, and qualitative and semi-quantitative prediction results are also more reliable.

### BRIEF DESCRIPTION OF THE DRAWINGS

FIG. 1 is a flowchart of hierarchical structural alert identification and high-throughput screening based on nuclear receptor molecular initiation events, taking AR as an example;

FIG. 2 is a diagram showing the prediction results of a training set and a test set of an active/inactive module by AR, ERα/β, GR, MR, PR, RARα/β/γ, TRα/β and VDR;

FIG. 3 is a diagram showing the prediction results of a training set and a test set of an agonist/A-Anta/antagonist module by AR, ERα/β, GR, MR, PR, RARα/β/γ, TRα/β and VDR;

FIG. 4 is a prediction flowchart of hierarchical screening of potential active endocrine disruptors based on structural alerts, taking ERα as an example;

FIG. 5 is a diagram showing the structural alerts of estrogen receptor alpha (ERα);

FIG. 6 is a diagram showing the structural alerts of androgen receptor (AR);

FIG. 7 is a diagram showing the structural alerts of estrogen receptor beta (ERβ);

FIG. 8 is a diagram showing the structural alerts of glucocorticoid receptor (GR);

FIG. 9 is a diagram showing the structural alerts of progesterone receptor (PR);

FIG. 10 is a diagram showing the structural alerts of mineralocorticoid receptor (MR);

FIG. 11 is a diagram showing the structural alerts of retinoic acid receptor alpha (RARα);

FIG. 12 is a diagram showing the structural alerts of retinoic acid receptor beta (RARβ);

FIG. 13 is a diagram showing the structural alerts of retinoic acid receptor gamma (RARγ);

FIG. 14 is a diagram showing the structural alerts of thyroid hormone receptor alpha (TRα);

FIG. 15 is a diagram showing the structural alerts of thyroid hormone receptor beta (TRβ);

FIG. 16 is a diagram showing the structural alerts of vitamin D receptor (VDR).

### DETAILED DESCRIPTION

In order to make the objectives, technical solutions and advantages of the examples of the present invention clearer, the following clearly and completely describes the technical solutions in the examples of the present invention with reference to accompanying drawings in the examples of the present invention. Apparently, the described examples are some rather than all of the examples of the present invention. Moreover, the various examples are not relatively independent and can be combined with each other according to needs, thus achieving better effects. Therefore, the following detailed description of the examples of the present invention provided in the accompanying drawings is not intended to limit the scope of the claimed present invention, but merely represents selected examples of the present invention. All other examples obtained by a person of ordinary skill in the art based on the examples of the present invention without creative efforts shall fall within the protection scope of the present invention.

In order to further understand the content of the present invention, the present invention will be described in detail in conjunction with the accompanying drawings and examples.

### Example 1

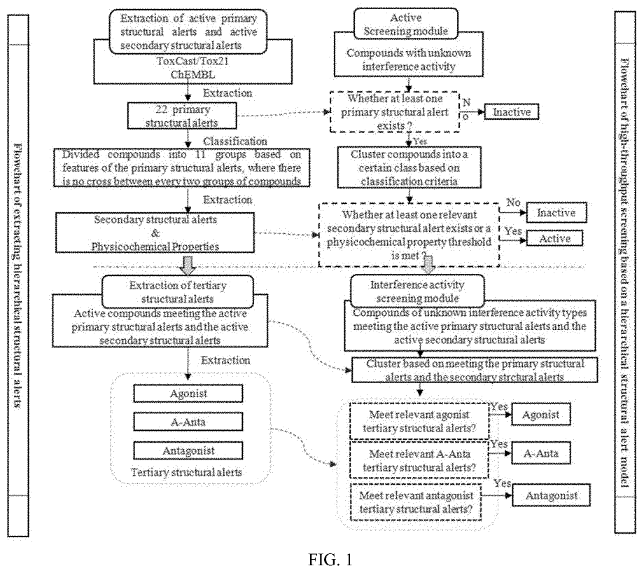

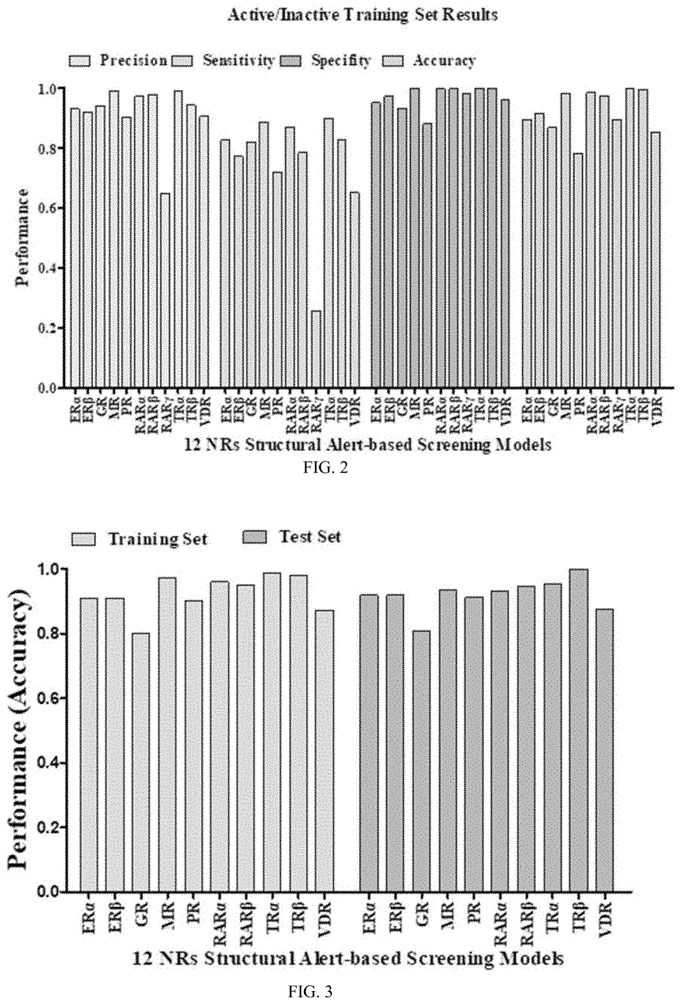

Referring to FIGS. 1-3, FIG. 1 is a flowchart of hierarchical structural alert identification and high-throughput screening based on nuclear receptor molecular initiation events, taking AR as an example; FIG. 2 is a diagram showing the prediction results of a training set and a test set of an active/inactive module by AR, ERα/β, GR, MR, PR, RARα/β/γ, TRα/β and VDR; and FIG. 3 is a diagram showing the prediction results of a training set and a test set of an agonist/A-Anta/antagonist module by AR, ERα/β, GR, MR, PR, RARα/β/γ, TRα/β and VDR.

According to the model for high-throughput screening of endocrine disruptors provided by this example, primary structural alerts, secondary structural alerts and tertiary structural alerts of compounds are extracted according to a nuclear receptor first. Specifically, compounds (as shown in Table 1) which have three kinds of in vitro experimental data of a competitive binding experiment, a reporter gene experiment and a cytotoxicity experiment are first acquired from public databases (ToxCast/Tox21, ChEMBL), and an obtained compound data set is classified; a method for randomly dividing the compound data set into a training set and a test set may utilize the partitioning mode in KNIME (https://www.knime.com/) to select Relative[%] in the First partition, input a value of 60%-80%, and then select Draw randomly to randomly divide the data set into the training set and the test set, wherein the training set is used to extract hierarchical structural alerts to construct a high-throughput screening model, and the test set is used for external verification. The compounds can be divided into five categories: active, inactive, agonistic, antagonistic and A-Anta, where active means that there is competitive binding activity and at least one reporter gene experimental activity, and both activity values must be greater than a cytotoxicity experimental value. For example, there is a compound Flutamide with CAS number 13311-84-7, its competitive binding experimental activity value is 6.39, the antagonistic androgen reporter gene experimental activity value is 4.7, and the cytotoxicity experimental value is 4.4. The cytotoxicity data is less than both the competitive binding activity value and the antagonistic androgen reporter gene activity value, proving that the compound is an active compound. Inactive means that there is neither competitive binding activity nor reporter gene activity (including agonistic androgen reporter gene experiment and antagonistic androgen reporter gene experiment). For example, there is a compound 1-Chloro-4-nitrobenzene with CAS number 100-00-5, its competitive binding activity value is 0, the agonistic androgen reporter gene experimental activity value is 0, and the antagonistic androgen reporter gene experimental activity value is 0, proving that the compound is inactive. Then the primary structural alerts of the compounds are extracted: according to the definitions of active and inactive compounds, the whole data set is divided into two parts of active compounds and inactive compounds, and then active primary structural alerts are extracted through substructure frequency analysis and substructure percentage analysis. Specific steps are as follows:

First of all, substructures contained in all compounds are calculated by using a PubChem fingerprint database in PaDEL-descriptor software. A specific method for calculating the substructures by using the PaDEL-descriptor software is to select Fingerprints from the Descriptors column in General, select Remove salt, Detect aromaticity and Standardize nitro groups in Standardize, and then check a Pubchemfingerpints molecular fingerprint database in Fingerprints to calculate the substructures of all the compounds. Frequencies of all the substructures are obtained by using formula

$$Frequency of a fragment = \frac{( {N}_{fragment}^{A} \times {N}_{total} )}{( {N}_{fragment \_ total} \times {N}_{A} )} ,$$

and sorted from high to low. The first 80-120 structural fragments are used as a threshold to obtain high frequency structural fragments. Based on a substructure percentage analysis algorithm with specific formula

$$Percentage of a fragment = \frac{{N}_{fragment}^{A}}{{N}_{A}} or \frac{{N}_{fragment}^{I}}{{N}_{I}} ,$$

a pheatmap algorithm in the R language is used to obtain structural fragments that can not only describe active compounds but also exclude inactive compounds, which are called primary structural alerts. Finally, the primary structural alerts are denoted by SMARTS strings. The pheatmap algorithm in the R language is as follows:

library (pheatmap)

tot<-read.csv(“C:/Agonist.csv”, row.names=1)

pheatmap(tot,cluster_cols=FALSE,border_color=NA,fontsize_row=3)

wherein the above-mentioned Frequency of a fragment denotes substructure frequency; Percentage of a fragment denotes substructure percentage; N_{fragment}^{A} is the number of active compounds containing the structural fragment; N total is the total number of compounds in a whole data set; N_{fragment_total} is the total number of compounds containing the structural fragment; N_{A} is the total number of active compounds in the data set; N_{fragment}^{I} is the total number of inactive compounds containing the structural fragment; and N_{I} is the total number of inactive compounds in the data set.

TABLE 1

| | | |
|---|---|---|
| | | Experiment |
| Experiment name | Species | type |

ERα

| | | | |
|---|---|---|---|
| ToxCast/ | NVS_NR_hER | Homo | Competitive |
| Tox21 | | sapiens | binding |
| | TOX21_ERa_BLA_Agonist_ratio | | receptor gene |
| | TOX21_ERa_LUC_BG1_Agonist | | receptor gene |
| | TOX21_ERa_BLA_Antagonist_ratio | | receptor gene |
| | TOX21_ERa_LUC_BG1_Antagonist | | receptor gene |
| | NCCT_HEK293T_CellTiterGLO | | cytotoxicity |
| ChEMBL | Binding affinity | Homo | B |
| | Agonist/Antagonist | sapiens | F |

ERβ

| | | | |
|---|---|---|---|
| ToxCast/ | NVS_NR_hER | Homo | Competitive |
| Tox21 | | sapiens | binding |
| | ATG_ERb_TRANS2_up | | receptor gene |
| | ATG_ERb_TRANS2_dn | | receptor gene |
| | NCCT_HEK293T_CellTiterGLO | | cytotoxicity |
| ChEMBL | Binding affinity | Homo | B |
| | Agonist/Antagonist | sapiens | F |

AR

| | | | |
|---|---|---|---|
| ToxCast/ | NVS_NR_hAR | Homo | Competitive |
| Tox21 | | sapiens | binding |
| | Tox21_AR_BLA_Agonist_ratio | | receptor gene |
| | Tox21_AR_BLA_Antagonist_ratio | | receptor gene |
| | NCCT_HEK293T_CellTiterGLO | | cytotoxicity |
| ChEMBL | Binding affinity | Homo | B |
| | Agonist/Antagonist | sapiens | F |

GR

| | | | |
|---|---|---|---|
| ToxCast/ | NVS_NR_hGR | Homo | Competitive |
| Tox21 | | sapiens | binding |
| | TOX21_GR_BLA_Agonist_ratio | | receptor gene |
| | TOX21_GR_BLA_Antagonist_ratio | | receptor gene |
| | NCCT_HEK293T_CellTiterGLO | | cytotoxicity |
| ChEMBL | Binding affinity | Homo | B |
| | Agonist/Antagonist | sapiens | F |

MR

| | | | |
|---|---|---|---|
| ToxCast/ | ATG_MR_TRANS2_dn | Homo | receptor gene |
| Tox21 | ATG_MR_TRANS2_up | sapiens | receptor gene |
| | NCCT_HEK293T_CellTiterGLO | | cytotoxicity |
| ChEMBL | Binding affinity | Homo | B |
| | Agonist/Antagonist | sapiens | F |

PR

| | | | |
|---|---|---|---|
| ToxCast/ | NVS_NR_hPR | Homo | Competitive |
| Tox21 | | sapiens | binding |
| | ATG_PR_TRANS2_dn | | receptor gene |
| | ATG_PR_TRANS2_up | | receptor gene |
| | NCCT_HEK293T_CellTiterGLO | | cytotoxicity |
| ChEMBL | Binding affinity | Homo | B |
| | Agonist/Antagonist | sapiens | F |

RARα

| | | | |
|---|---|---|---|
| ToxCast/ | ATG_RARa_TRANS_up | Homo | receptor gene |
| Tox21 | ATG_RARa_TRANS_dn | sapiens | receptor gene |
| | NCCT_HEK293T_CellTiterGLO | | cytotoxicity |
| ChEMBL | Binding affinity | Homo | B |
| | Agonist/Antagonist | sapiens | F |

RARβ

| | | | |
|---|---|---|---|
| ToxCast/ | ATG_RARb_TRANS_dn | Homo | receptor gene |
| Tox21 | ATG_RARb_TRANS_up | sapiens | receptor gene |
| | NCCT_HEK293T_CellTiterGLO | | cytotoxicity |
| ChEMBL | Binding affinity | Homo | B |
| | Agonist/Antagonist | sapiens | F |

RARγ

| | | | |
|---|---|---|---|
| ToxCast/ | ATG_RARg_TRANS_up | Homo | receptor gene |
| Tox21 | ATG_RARg_TRANS_dn | sapiens | receptor gene |
| | NCCT_HEK293T_CellTiterGLO | | cytotoxicity |
| ChEMBL | Binding affinity | Homo | B |
| | Agonist/Antagonist | sapiens | F |

TRα

| | | | |
|---|---|---|---|
| ToxCast/ | TOX21_TR_LUC_GH3_Agonist | Homo | receptor gene |
| Tox21 | TOX21_TR_LUC_GH3_Antagonist | sapiens | receptor gene |
| | NCCT_HEK293T_CellTiterGLO | | cytotoxicity |
| ChEMBL | Binding affinity | Homo | B |
| | Agonist/Antagonist | sapiens | F |

TRβ

| | | | |
|---|---|---|---|
| ToxCast/ | TOX21_TR_LUC_GH3_Agonist | Homo | receptor gene |
| Tox21 | TOX21_TR_LUC_GH3_Antagonist | sapiens | receptor gene |
| | NCCT_HEK293T_CellTiterGLO | | cytotoxicity |
| ChEMBL | Binding affinity | Homo | B |
| | Agonist/Antagonist | sapiens | F |

VDR

| | | | |
|---|---|---|---|
| ToxCast/ | TOX21_VDR_BLA_agonist_ratio | Homo | receptor gene |
| Tox21 | TOX21_VDR_BLA_antagonist_ratio | sapiens | receptor gene |
| | NCCT_HEK293T_CellTiterGLO | | cytotoxicity |
| ChEMBL | Binding affinity | Homo | B |
| | Agonist/Antagonist | sapiens | F |

Then the secondary structural alerts are extracted from the compounds:

Based on structural features of the primary structural alerts, the active/inactive compounds containing the primary structural alerts are classified first (there is no cross between every two groups of compounds). A specific method for classifying the compounds includes: taking aromatic ring bonds as important structural features, taking polar atoms (such as oxygen atoms and nitrogen atoms) as secondary important structural features, thereby taking the primary structural alerts containing the polar atoms and the aromatic ring bonds as Type 1 (the first category); taking the primary structural alerts containing only the polar atoms (such as the oxygen atoms and the nitrogen atoms) and not containing the aromatic ring bonds as Type 2 (the second category), and finally taking any carbon chains as Type 3 (the third category). The compounds are divided into three categories according to the contained primary structural alerts, and there is no intersection in each category.

Then, for each group of compounds, feature structural alerts are extracted by using SARpy software. The feature structural alerts are artificially verified and irrelevant small fragment sets are screened out, and the remaining key structural alerts are the secondary structural alerts. Meanwhile, if there is a significant deviation between the number of active compounds and the number of inactive compounds and effective structural alerts cannot be extracted, then the active/inactive compounds are distinguished by using relevant physicochemical properties. Specifically, a method for identifying the feature secondary structural alerts of each type of compounds respectively comprises: setting the activity of active compounds in a training set to 1, setting the activity of inactive compounds to 0, forming a CSV file by using their names, structure SMILES numbers and set activity values, importing the CSV file into SARpy software (http://sarpy.sourceforge.net/), selecting corresponding SMILES column and ACTIVITY attribution in a Get a DATASET module, and then setting Set numeric threshold to 0; selecting LOW<=0<HIGH in Select the desired splitting (threshold belongs to “LOW” or “HIGH” class), checking Binarize (optional) to set activity >0 to be ACTIVE and set activity <=0 to be INACTIVE, and clicking a Loading button to import the data set; in a Get a RULESET module, selecting ACTIVE in Select the TARGET activity class, meanwhile setting other parameters respectively: Customize single alert precision (Auto: MAX), HIGH SPECIFICITY (minimize false positive), clicking EXTRACT and VALIDATE, and extracting the secondary structural alerts;

finally denoting the secondary structural alerts by SMARTS strings. A specific method for calculating physicochemical properties is to use PaDEL-descriptor software to select 1D&2D and 3D in the Descriptors column in General, select Remove salt, Detect aromaticity and Standardize nitro groups in Standardize, then select all in 1D&2D and all in 3D, and calculate the physicochemical properties of compounds.

Step (6) extraction of the tertiary structural alerts: based on the screening of the primary structural alerts and the secondary structural alerts, the active compound and the inactive compound are distinguished, and then the tertiary structural alerts are extracted based on the active compounds meeting the secondary structural alerts to predict the disrupting activity of the active compounds. The active compounds in each group are first divided into three types depending on in vitro experimental activity results, namely, agonist, A-Anta and antagonist, then analysis is performed based on the corresponding secondary structural alerts, the secondary structural alerts with significant distinction between compounds with different disrupting activity are defined as the tertiary structural alerts, and tertiary structural alerts are further extracted from the secondary structural alerts and relevant compounds thereof without significant distinction between compounds with different disrupting activity by using SARpy software. Finally, the agonist/A-Anta/antagonist three-level structural alerts are denoted by SMARTS strings. It is worth noting that agonist means that in the defined active compounds, there is agonistic reporter gene experimental activity, but there is no antagonistic reporter gene experimental activity, that is, the agonistic reporter gene experimental activity is greater than 0 and greater than cytotoxicity, the antagonistic reporter gene experimental activity is equal to 0 or less than or equal to the cytotoxicity, and then the compounds are agonistic disruptors.

A-Anta means that in the defined active compounds, there are agonistic reporter gene experimental activity and antagonistic reporter gene experimental activity, that is, the agonistic reporter gene experimental activity is greater than 0 and greater than cytotoxicity, the antagonistic reporter gene experimental activity is greater than 0 and greater than cytotoxicity, and then the compounds are A-Anta disruptors.

Antagonist means that in the defined active compounds, there is no agonistic reporter gene experimental activity, but there is antagonistic reporter gene experimental activity, that is, the agonistic reporter gene experimental activity is equal to 0 or less than or equal to cytotoxicity, the antagonistic reporter gene experimental activity is greater than cytotoxicity, and then the compounds are antagonistic disruptors.

It is worth noting that significant distinction means that statistically a certain structural alert basically only exists in a certain active compound (agonist, antagonist or A-Anta), and this structural alert is taken as a feature structural alert of such active compound. For example, if only the agonist compound has a structural alert A and two other compounds with disrupting activity do not have the structural alert A, then this structural alert A is taken as the feature structural alert of the agonistic compounds. A method for determining a significant difference is to use the One-way ANOVA (and nonparametric) statistical algorithm for analysis. When the calculated p value is less than 0.05, it is considered that there is a statistically significant difference.

A method for selecting a tertiary structural alert data set and extracting relevant tertiary structural alerts comprises: extracting tertiary structural alerts by taking active compounds meeting the primary structural alerts and the relevant secondary structural alerts in the training set as a new training set. The active compounds in each group are first divided into three types (agonistic disruptors, antagonistic disruptors and A-Anta disruptors) depending on disrupting activity, and then the names, SMILES numbers and activity values (uniformly set to 1) of the active compounds are used to construct a CSV file and the CSV file is imported into SARpy software; in Load Ruleset of a Get a RULESET module, secondary structural alert SMARTS information and Taget (ACTIVE) information are imported in text file format; in a Predict and Validate module, Predict and Validate are clicked, and then Save predictions is clicked to obtain the secondary structural alert information contained in the active compounds. Then in the predicted results, ACTIVE is set to 1 and None is set to 0, and cluster analysis is performed by using pheatmap algorithm in the R language.

library (pheatmap)

tot<-read.csv(“C:/Agonist.csv”,row.names=1)

pheatmap(tot,cluster_cols=FALSE,border_color=NA,fontsize_row=3)

According to thermographic analysis, the only feature secondary structural alert that exists under a certain disrupting activity condition is set as the relevant tertiary structural alert, and meanwhile, high-throughput screening is carried out on disrupting compounds (Tier 1). Then, for the active compounds whose feature secondary structural alert cannot be distinguished, SARpy software is used to extract the relevant feature tertiary structural alert by adopting the same parameter settings as the preceding step (Tier 2). Through Tier 1 and Tier 2 steps, extraction of the tertiary structural alerts of nuclear receptor-mediated endocrine disruptors is completed.

Then, the extracted primary structural alerts, secondary structural alerts and tertiary structural alerts form a nuclear receptor high-throughput screening model. It needs to be noted that the nuclear receptor can only be AR, ERα, ERβ, GR, MR, PR, RARα, RARβ, RARγ, TRα, TRβ and VDR (as shown in Table 2).

TABLE 2
ERα

| | | Active | | | |
|---|---|---|---|---|---|
| | | | Agonist + | | |
| | | Agonist | antagonist | Antagonist | Inactive |
| ToxCast/ | Training | 338 | 155 | 290 | 1186 |
| Tox21 | set | | | | |
| ChEMBL | Test set | 86 | 39 | 74 | 297 |
| | Sum | 424 | 194 | 364 | 1483 |
| | | Active | | | |
| | | Agonist | Agonist | Agonist | Inactive |

ERβ

| | | | | | |
|---|---|---|---|---|---|
| ToxCast/ | Training | 319 | 62 | 108 | 1696 |
| Tox21 | set | | | | |
| ChEMBL | Test set | 79 | 15 | 27 | 423 |
| | Sum | 398 | 77 | 135 | 2119 |

AR

| | | | | | |
|---|---|---|---|---|---|
| ToxCast/ | Training | 107 | 234 | 345 | 1589 |
| Tox21 | set | | | | |
| | Test set | 19 | 59 | 94 | 398 |
| ChEMBL | Sum | 126 | 293 | 439 | 1987 |

GR

| | | | | | |
|---|---|---|---|---|---|
| ToxCast/ | Training | 325 | 326 | 41 | 1494 |
| Tox21 | set | | | | |
| | Test set | 82 | 83 | 11 | 374 |
| ChEMBL | Sum | 407 | 409 | 52 | 1868 |

MR

| | | | | | |
|---|---|---|---|---|---|
| ToxCast/ | Training | 30 | 24 | 348 | 2629 |
| Tox21 | set | | | | |
| | Test set | 7 | 6 | 86 | 657 |
| ChEMBL | Sum | 37 | 30 | 434 | 3286 |

PR

| | | | | | |
|---|---|---|---|---|---|
| ToxCast/ | Training | 163 | 116 | 131 | 688 |
| Tox21 | set | | | | |
| | Test set | 40 | 29 | 32 | 172 |
| ChEMBL | Sum | 203 | 145 | 163 | 860 |

RARα

| | | | | | |
|---|---|---|---|---|---|
| ToxCast/ | Training | 392 | 20 | 85 | 2387 |
| Tox21 | set | | | | |
| | Test set | 99 | 5 | 23 | 597 |
| ChEMBL | Sum | 491 | 25 | 108 | 2984 |

RARβ

| | | | | | |
|---|---|---|---|---|---|
| ToxCast/ | Training | 244 | — | 127 | 2517 |
| Tox21 | set | | | | |
| | Test set | 63 | — | 33 | 630 |
| ChEMBL | Sum | 307 | — | 160 | 3147 |

RARγ

| | | | | | |
|---|---|---|---|---|---|
| ToxCast/ | Training | 226 | — | 75 | 2548 |
| Tox21 | set | | | | |
| | Test set | 58 | — | 20 | 637 |
| ChEMBL | Sum | 284 | — | 95 | 3185 |

TRα

| | | | | | |
|---|---|---|---|---|---|
| ToxCast/ | Training | 79 | — | 21 | 5442 |
| Tox21 | set | | | | |
| ChEMBL | Test set | 20 | — | 5 | 1360 |
| | Sum | 99 | — | 26 | 6802 |

TRβ

| | | | | | |
|---|---|---|---|---|---|
| ToxCast/ | Training | 91 | — | 30 | 5442 |
| Tox21 | set | | | | |
| | Test set | 23 | — | 7 | 1360 |
| ChEMBL | Sum | 114 | — | 37 | 6802 |

VDR

| | | | | | |
|---|---|---|---|---|---|
| ToxCast/ | Training | 262 | 32 | 434 | 5274 |
| Tox21 | set | | | | |
| | Test set | 67 | 9 | 110 | 1319 |
| ChEMBL | Sum | 329 | 41 | 544 | 6593 |

A method for high-throughput screening of endocrine disruptors provided by this example adopts the foregoing model for high-throughput screening of endocrine disruptors to screen target compounds, and the method comprises the following steps:

Step 1: Acquisition of Structural Data

Chemical structures of target compounds without relevant activity data are selected from a public database, and denoted by SMILES numbers; specifically, the chemical structures of the target compounds without relevant activity data are first searched and selected from the public database, which includes ChEMBL, PubChem, ChemicalBook, Chemspider, and the like. The chemical structures of the target compounds are denoted by the SMILES numbers, and then the SMILES numbers of the target compounds are imported into ChemBioDraw Ultra 14.0 software for examination. If there are no errors in the structures of the target compounds, the target compounds can be used for disrupting activity prediction. It is worth noting that the target compounds are organic compounds and cannot be compounds such as mixtures and heavy metals, because the high-throughput screening model is only used for the prediction of disrupting activity of organics.

Step 2: Determination of a Target Nuclear Receptor

A target nuclear receptor is determined according to the disrupting activity of the target compounds to be predicted; that is, an appropriate target nuclear receptor is selected according to the disrupting activity to be predicted, wherein the target nuclear receptor can only be AR, ERα/β, GR, MR, PR, RARα/β/γ, TRα/β and VDR.

Step 3: Matching of Primary Structural Alerts

A target nuclear receptor high-throughput screening model is selected according to the determined target nuclear receptor, molecular fingerprints of the target compounds are calculated by using a PubChem fingerprints molecular fingerprint database of PaDEL-Descriptor software, and a calculated result is matched with primary structural alerts of the target nuclear receptor high-throughput screening model. Specifically, the corresponding target nuclear receptor high-throughput screening model is selected first according to the determined target nuclear receptor, and primary structural alerts of the target nuclear receptor high-throughput screening model are denoted by SMARTS strings, which are used to match the chemical structures of the target compounds. ERα is taken as an example. When the target nuclear receptor is ERα, there are 18 primary structural alerts, all denoted by SMARTS strings (e.g., C-C:C-O-[#1]). The SMILE numbers of the target compound are imported into PaDEL-descriptor software, Fingerprints is selected in the Descriptors column in General; Remove salt, Detect aromaticity and Standardize nitro groups are selected in Standardize, and a Pubchemfingerpints molecular fingerprint database is checked in Fingerprints to calculate PubChem fingerprints substructures of the target compound. The obtained molecular fingerprints of the target compounds are compared with a primary structural alert database of the target compound; when the target compound meet at least one primary structural alert, it is determined that the target compound has an endocrine regulation process that potentially disrupts the regulation of the target nuclear receptor, and it is determined that the target compound is active compound. In contrast, when the target compound does not meet any one of the primary structural alerts, it is determined that the target compound is inactive compound.

Step 4: Matching of Secondary Structural Alerts

The target compounds meeting the primary structural alerts are grouped based on the primary structural alerts, and the target compounds are matched with the secondary structural alerts of the target nuclear receptor high-throughput screening model by using SARpy software; specifically, the primary structural alerts of the target nuclear receptor high-throughput screening model are grouped first, and the primary structural alerts containing polar atoms and aromatic ring bonds are taken as Type 1; the primary structural alerts containing aromatic bonds are taken as Type 2; the primary structural alerts of carbon chains containing oxygen atoms are taken as Type 3; finally the primary structural alerts of any carbon chains without oxygen atoms and aromatic bonds are taken as Type 4; wherein the priorities of Type 1, Type 2, Type 3 and Type 4 are successively decreased, the primary structural alerts of the target compounds are matched with the primary structural alerts in each group, and then the target compounds are classified from the group with successful matching to the group with the highest priority. For further illustration, ERα is taken as an example, and its 18 primary structural alerts are divided into four types: structural alerts containing oxygen atoms and aromatic bonds (Type 1), structural alerts containing aromatic bonds (Type 2), carbon chains containing oxygen atoms (Type 3) and any carbon chains without oxygen atoms and aromatic bonds (Type 4). The compound is divided into three independent large groups according to the descending order of priorities, and there is no intersection between the large groups. For example, the target compound has four types of primary structural alerts, namely, Type 1, Type 2, Type 3 and Type 4, the target compound is classified into Type 1 group according to the priority decreasing rule for subsequent matching of specific secondary structural alerts in Type 1 group.

Then, SARpy software is used to match the target compounds with the secondary structural alerts of the target nuclear receptor high-throughput screening model, i.e. matching of the specific secondary structural alerts within the group is performed on the grouped target compounds. For detailed description, the Type 1 group of ERα is taken as an example. When the target compound is classified into this group, the target compound needs to be matched with the corresponding 27 secondary structural alerts (all denoted by SMARTS strings, e.g., CCCC(C)c1ccccc1). A matching method comprises setting the activity of the target compound to 1, forming a CSV file by using the name, structure SMILES number and set activity value of the target compound, importing the CSV file into SARpy software, selecting corresponding SMILES column and ACTIVITY attribution in a Get a DATASET module, and then setting Set numeric threshold to 0; selecting LOW<=0<HIGH in Select the desired splitting (threshold belongs to “LOW” or “HIGH” class), checking Binarize (optional) to set activity >0 to be ACTIVE, set activity <=0 to be INACTIVE, and clicking a Loading button to import the data set. In Load Ruleset in a Get a RULESET module, SMARTS strings information and Target(ACTIVE) information of the 27 secondary structural alerts are imported in text file format. In a Predict and Validate module, Predict and Validate are clicked, and then Save predictions is clicked to obtain the result of whether the target compound meets the secondary structural alerts and the relevant secondary structural alert information.

Step 5: Matching of Tertiary Structural Alerts

Target compounds meeting both the primary structural alerts and the secondary structural alerts are grouped based on the secondary structural alerts, and matched with tertiary structural alerts of the target nuclear receptor high-throughput screening model by using the SARpy software. Specifically, the primary structural alerts of the target nuclear receptor high-throughput screening model are grouped first, and the primary structural alerts containing polar atoms and aromatic ring bonds are taken as Type 1; the primary structural alerts containing aromatic bonds are taken as Type 2; the primary structural alerts of carbon chains containing oxygen atoms are taken as Type 3; finally the primary structural alerts of any carbon chains without oxygen atoms and aromatic bonds are taken as Type 4; wherein the priorities of Type 1, Type 2, Type 3 and Type 4 are successively decreased, the primary structural alerts of the target compounds are matched with the primary structural alerts in each group, and then the target compounds are classified from the group with successful matching to the group with the highest priority.

For example, the target compounds have three types of secondary structural alerts, namely, Type 1-1, Type 1-2 and Type 1-3, the compounds are classified into Type 1-1 group according to the priority decreasing rule for subsequent matching of specific tertiary structural alerts in Type 1-1 group, that is, matching with the tertiary structural alerts of the target nuclear receptor high-throughput screening model by using SARpy software. It should be noted that agonist means that disruptors have agonistic disruption and do not have antagonistic disruption; A-Anta means that the disruptors have agonistic disruption and antagonistic disruption; and antagonist means that the disruptors do not have agonistic disruption but have antagonistic disruption.

The matching of specific tertiary structural alerts in the group is further illustrated. The Type 1-1 group of ERα is taken as an example. After the target compound is subject to secondary classification into the Type 1-1 group, since the secondary structural alerts of Type 1-1 are owned only by disruptors with antagonistic activity, that is, after the target compound meets any one of the structural alerts in at least Type 1-1, it is determined that the disruption type of the target compound is antagonistic, and the matching of the tertiary disrupting structural alerts is no longer required. Take the Type 1-3 group as an example. There are five secondary structural alerts in the group. When the target compound does not meet the secondary structural alerts of Type 1-1 and Type 1-2, but meets at least one of the five secondary structural alerts of Type 1-3, it is determined that the target compound is an active compound and belongs to the Type 1-3 active compounds, thereby determining whether the target compound meets one of the four structural alerts in Type 1-3-1. When the target compound meets at least one of the four structural alerts, it is determined that the target compound is an A-Anta active disruptor; otherwise, the target compound belongs to an agonist active disruptor. A matching method comprises setting the activity of the target compounds to 1, forming a CSV file by using names, structure SMILES numbers and set activity values of the target compounds, importing the CSV file into SARpy software (http://sarpy.sourceforge.net/), selecting corresponding SMILES column and ACTIVITY attribution in a Get a DATASET module, and then setting Set numeric threshold to 0; selecting LOW<=0<HIGH in Select the desired splitting (threshold belongs to “LOW” or “HIGH” class), checking Binarize (optional) to set activity >0 to be ACTIVE, set activity <=0 to be INACTIVE, and clicking a Loading button to import the data set. In Load Ruleset in a Get a RULESET module, SMARTS strings information and Taget(A-Anta) information of four tertiary structural alerts of Type 1-3-1 are imported in text file format. In a Predict and Validate module, Predict and Validate are clicked, and then Save predictions is clicked to obtain the result of whether the target compounds meet the tertiary structural alerts and the relevant disrupting activity category information.

By constructing the model for high-throughput screening of endocrine disruptors and then matching the hierarchical structural alerts of the target compounds, the receptor competitive activity and A-Anta activity of the endocrine disruptors can be qualitatively determined; the structural alerts corresponding to different types of endocrine disruptors are identified by using three endocrine disruption mechanisms of agonist, A-Anta and antagonist, the relationship between the activity and the internal mechanism is successfully established, and magnitudes of the binding activity of the disruptors and the disrupting activity of some of the disruptors are semi-quantitatively predicted.

Step 6: Ligand-Receptor Binding Mode Analysis

Target compounds meeting the primary structural alerts, the secondary structural alerts and the tertiary structural alerts are selected through the target nuclear receptor high-throughput screening model, a pattern protein receptor crystal is selected from RCSB PDB Database based on the disrupting activity of the target compounds, molecular docking is performed by using Autodock vina software, and ligand-receptor binding mode analysis is performed by using Ligplus software. Particularly, the prediction of ligand-receptor binding modes is divided into an agonistic ligand-receptor binding mode, an A-Anta ligand-receptor binding mode, and an antagonistic ligand-receptor binding mode depending on disrupting activity categories. The binding mode consists of two aspects: hydrogen bonding between a target compound and a key polar amino acid in a target nuclear receptor pocket, and hydrophobic interaction between a target compound and a key non-polar amino acid in a target nuclear receptor pocket. For further illustration, the Type 1-1 group of ERα is taken as an example. When the target compound meets the tertiary structural alerts (feature secondary structure) Oc1ccc2C(N(CCc2c1)), the disrupting activity is antagonistic, and the predicted ligand-receptor binding mode is Arg394 & His524 (hydrogen bonding with two polar amino acids Arg394 and His524). That is, the compounds classified into Type 1-1 have the same primary, secondary and tertiary structural alerts, and have similar ligand-receptor binding mode with Arg394 & His524 as the main components.

It should be noted that ligand-receptor binding mode analysis is only applicable to three nuclear receptors which are AR, ERα and GR. That is, the target nuclear receptor high-throughput screening model can only be an AR high-throughput screening model, an ERα high-throughput screening model and a GR high-throughput screening model. Moreover, the ligand-receptor binding mode analysis is targeted at a first pocket and has no relation with a second pocket and other pockets. The first pocket (ligand bonding domain (LBD)) refers to the binding site of human nuclear receptor and natural hormone. This pocket is also the site where most environmental pollutants enter and disrupt the binding of the natural hormone and the nuclear receptor. However, in recent years, some researchers have also found that many pollutants may not enter this binding site and can also bind with the receptor in other cavities of the nuclear receptor, resulting in receptor allosterism, which affects abnormal up-regulation or down-regulation of transcription, and finally leads to endocrine disorder. According to the present invention, research is carried out on the first binding site, so that which amino acids the compounds interact with to produce disrupting activity can be further predicted, and then the generation mechanism of the disrupting activity can be predicted.

Step 7: Semi-Quantitative Prediction of Binding Activity and Disrupting Activity

Target compounds with the same structural alert have similar ligand-receptor binding mode and similar binding activity; when the binding activity is positively correlated with the disrupting activity, the target compounds are grouped based on the primary structural alerts, the secondary structural alerts and the tertiary structural alerts of the compounds, and the binding activity and the disrupting activity of the compounds are semi-quantitatively predicted. It is worth noting that the binding activity and the disrupting activity are standardized and classified in intensity by using formula

$$Activity value = - {log}_{10} \frac{{K}_{i} , {K}_{d} , {AC}_{50} , {IC}_{50} , {EC}_{50} ( uM )}{1000000} \text{:}$$

wherein Activity value denotes a value of activity intensity, Kᵢ denotes an inhibition constant, K_{d} denotes a dissociation constant, AC₅₀ denotes a median activity concentration, IC₅₀ denotes a median inhibitory concentration, EC₅₀ denotes a median effect concentration, uM denotes a micromolar weight, Activity value ≥7 denotes a strong activity intensity, 7>Activity value ≥6 denotes a medium activity intensity, and Activity value <6 denotes a weak activity intensity. It should be further illustrated that the semi-quantitative prediction of binding activity and disrupting activity is only applicable to three nuclear receptors which are AR, ERα, and GR. It is known that compounds with the same structural alert have similar ligand-receptor binding mode, which leads to similar binding activity. When the binding activity is positively correlated with the disrupting activity, the binding activity intensity is positively correlated with the disrupting activity intensity.

Therefore, the binding activity and disrupting activity of the compounds can be semi-quantitatively predicted by clustering the compounds in a specific group according to the existing primary, secondary and tertiary structural alerts of the target compounds. When there is no positive correlation between binding activity and disrupting activity, it is proved that besides ligand-receptor competitive binding, the recruitment process of co-regulatory factors is also a key step to determine the disrupting activity of compounds. For such compounds, prediction results give warning information. For further illustration, the Type 1-1 group of ERα is taken as an example. When the compound meets the tertiary structural alerts Oc1ccc2C(N(CCc2c1), the disrupting activity is antagonistic, its binding activity intensity is strong, and the resulting A-Anta disrupting activity is also strong.

In combination with steps 6 and 7, compounds with the same structural alert have similar ligand-receptor binding mode and similar binding activity; when the binding activity is positively correlated with the disrupting activity, the compounds are grouped according to the primary structural alerts, the secondary structural alerts and the tertiary structural alerts of the compounds, the binding activity and the disrupting activity of the compounds are semi-quantitatively predicted, and qualitative and semi-quantitative prediction results are also more reliable.

### Example 2

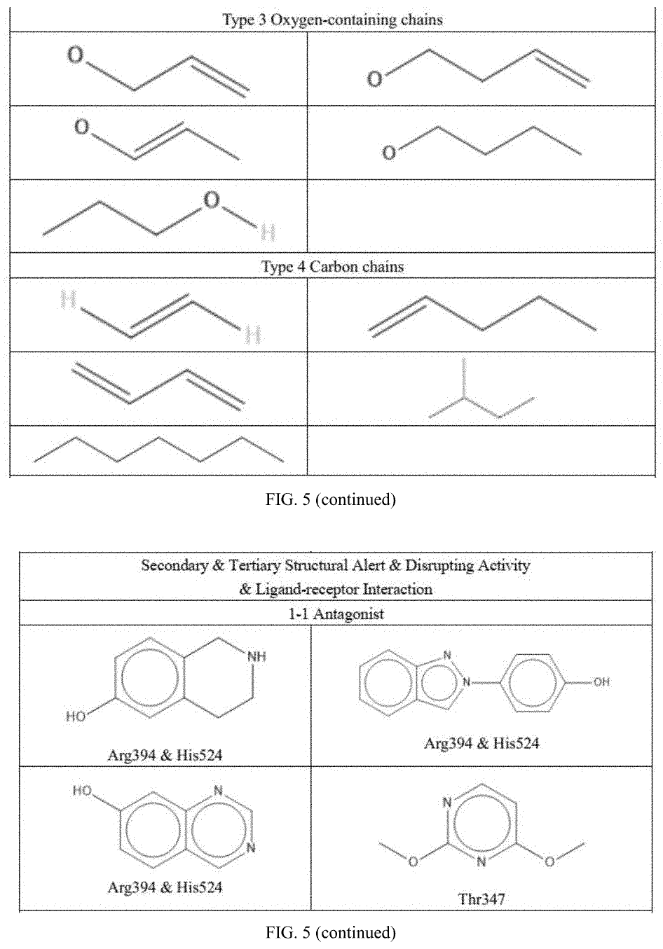

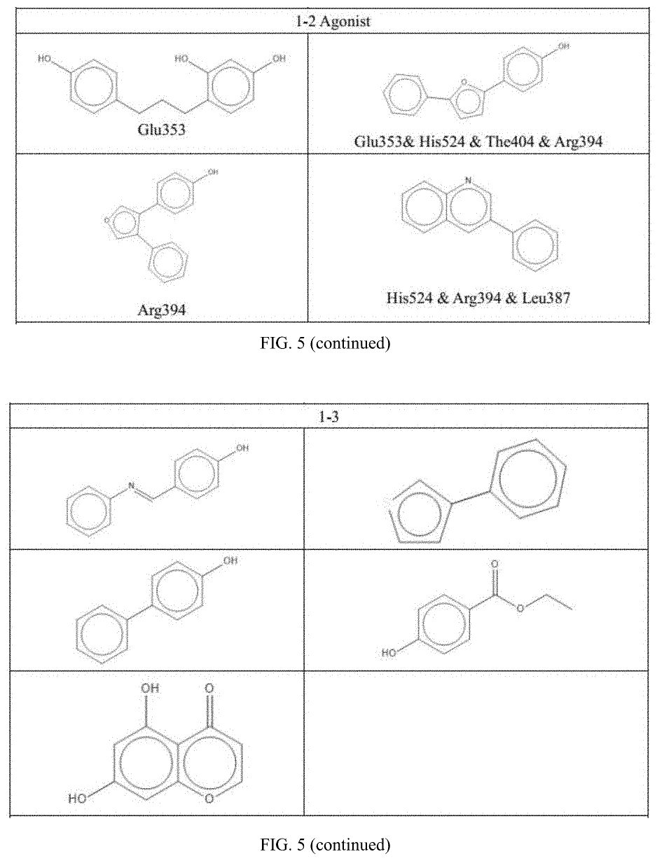

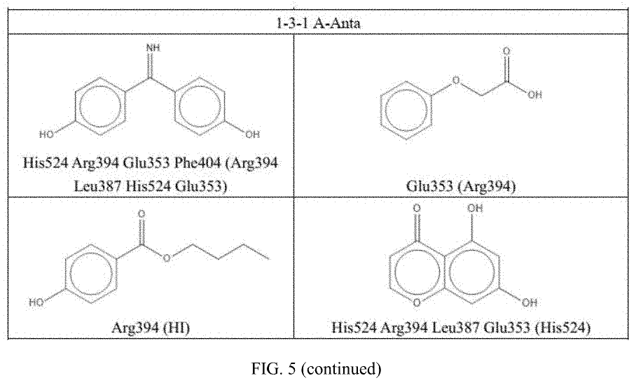

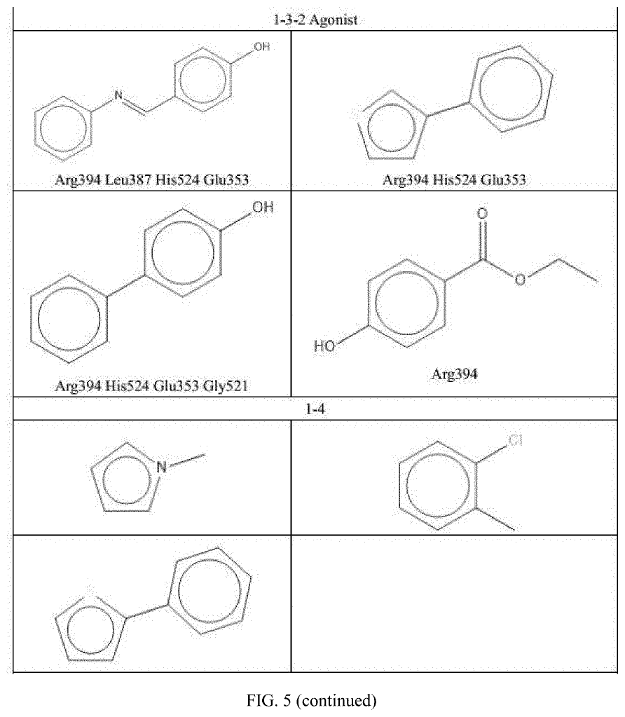

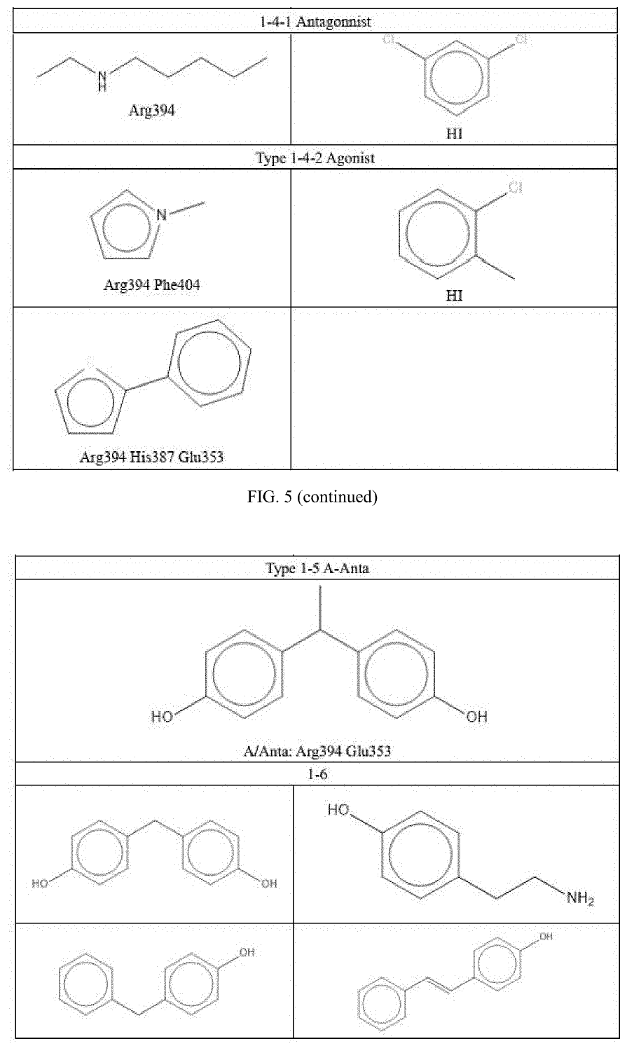

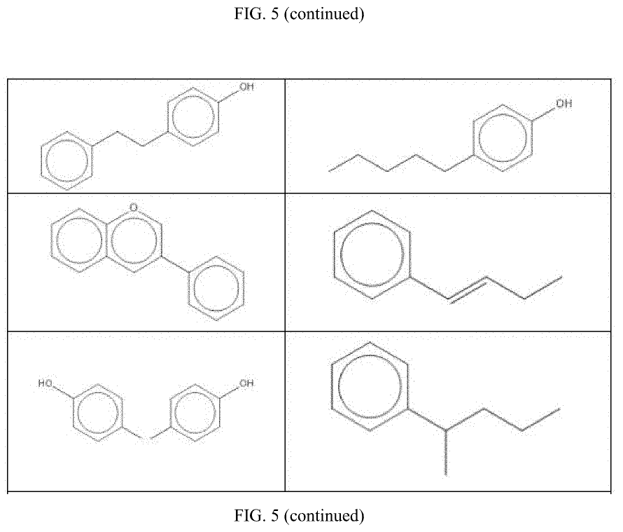

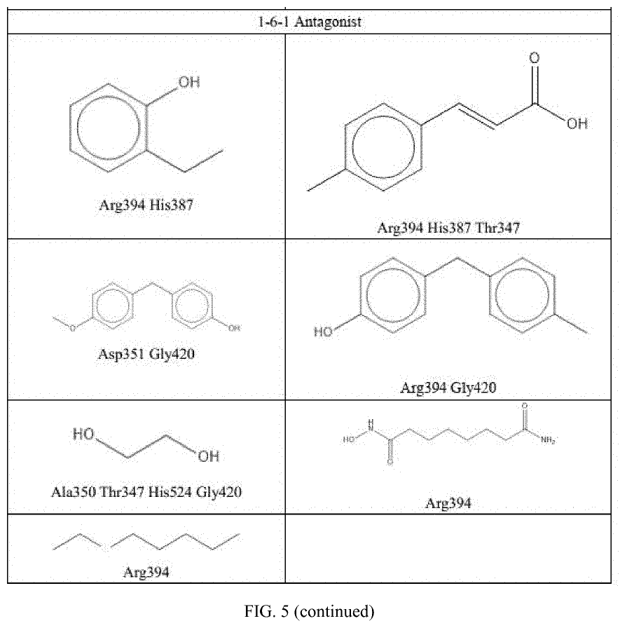

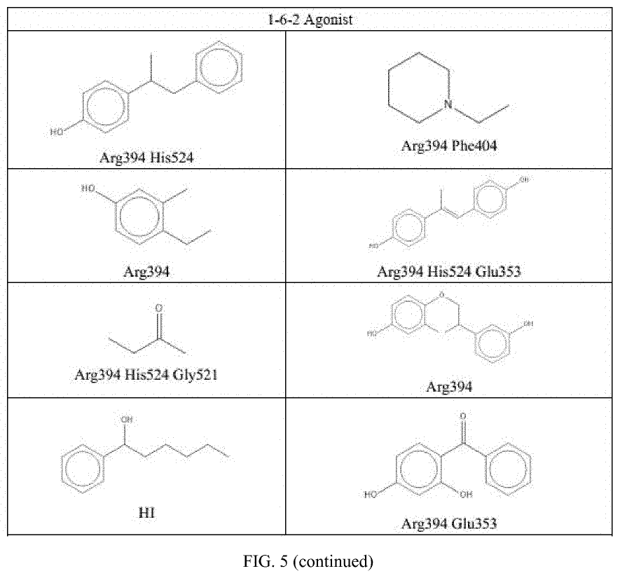

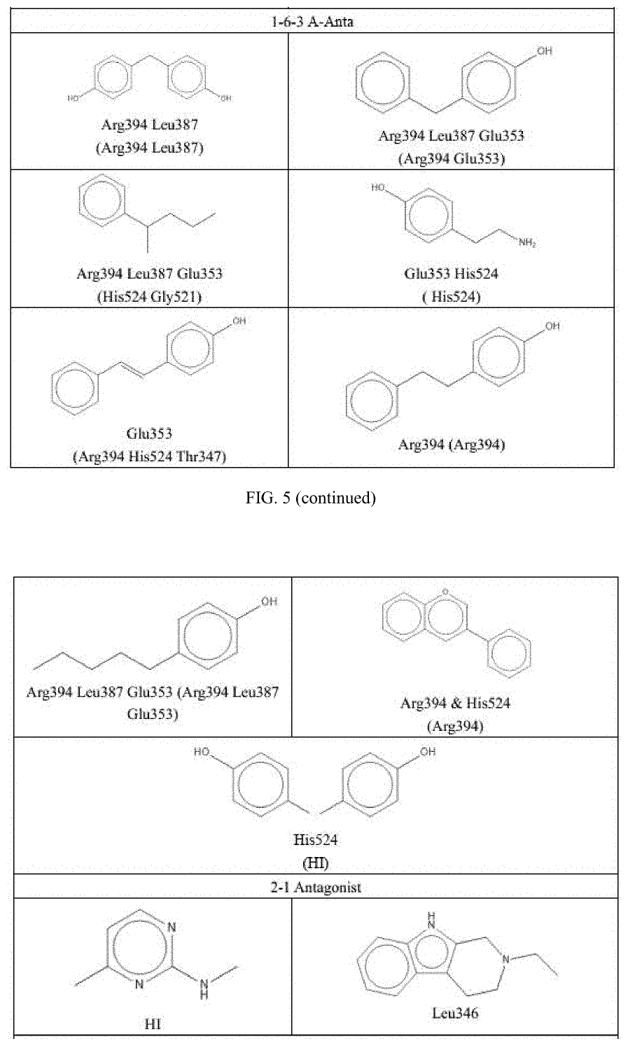

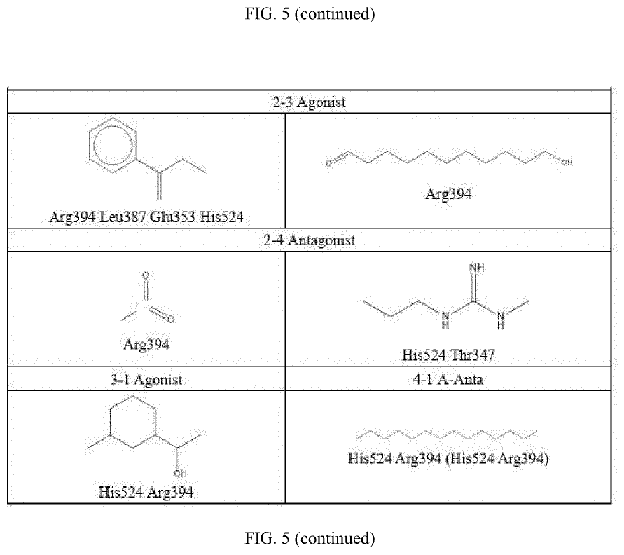

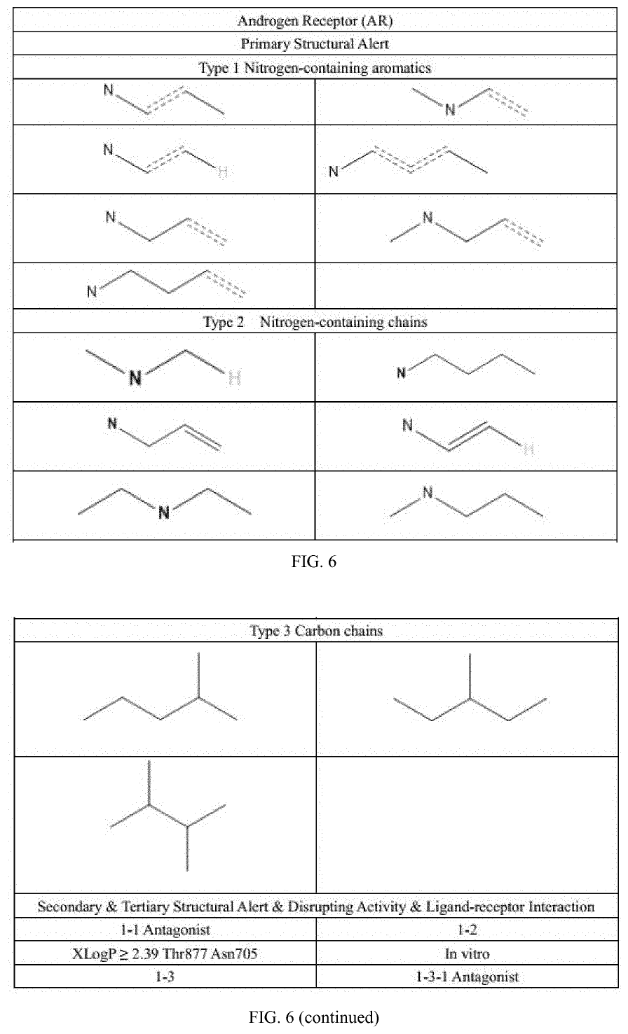

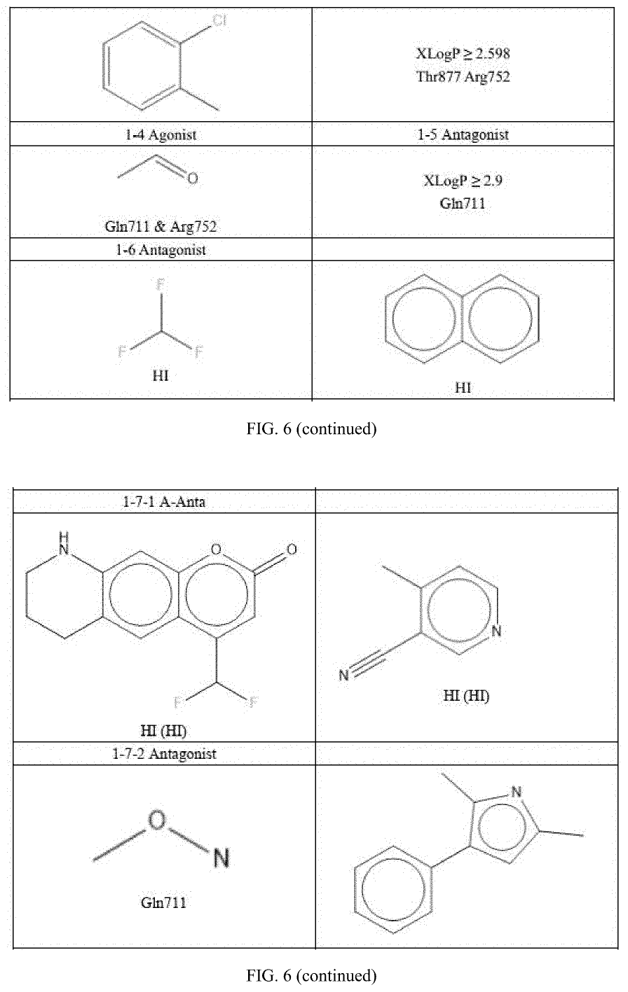

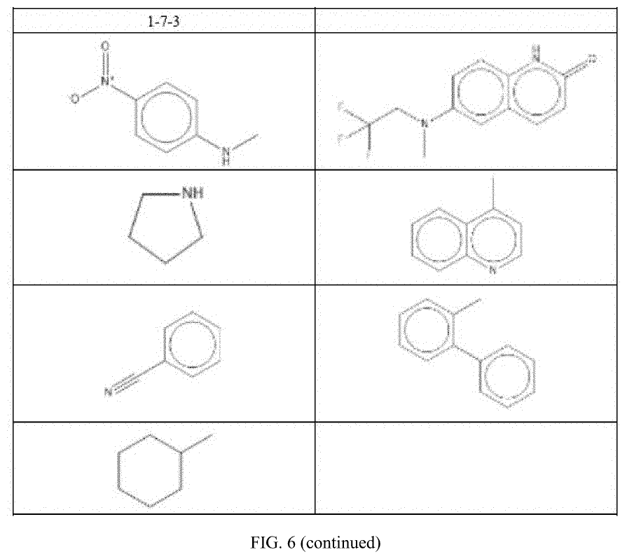

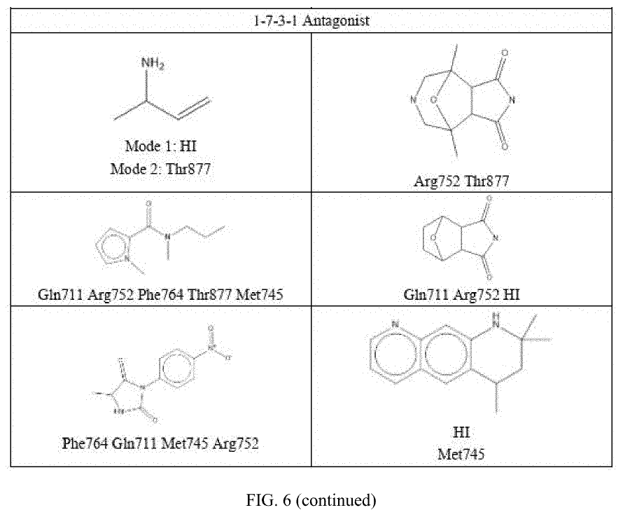

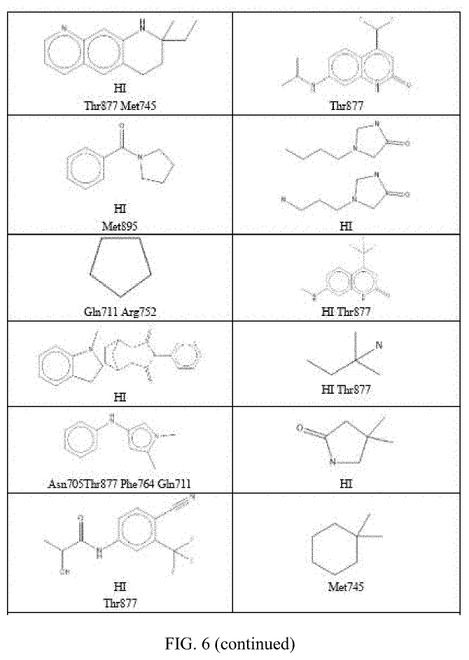

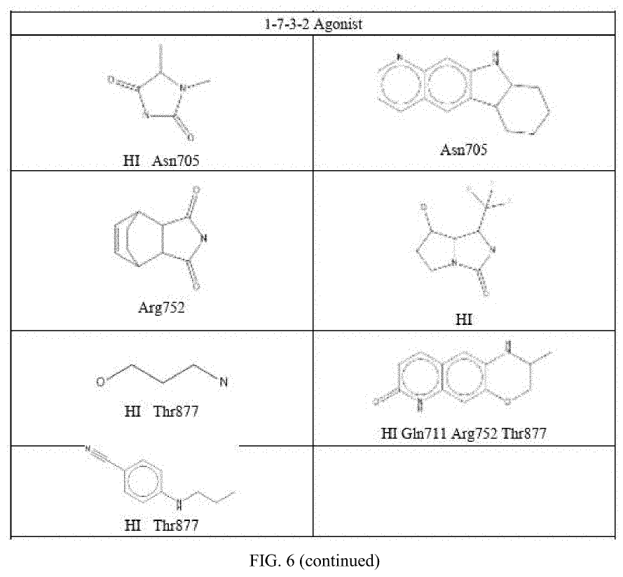

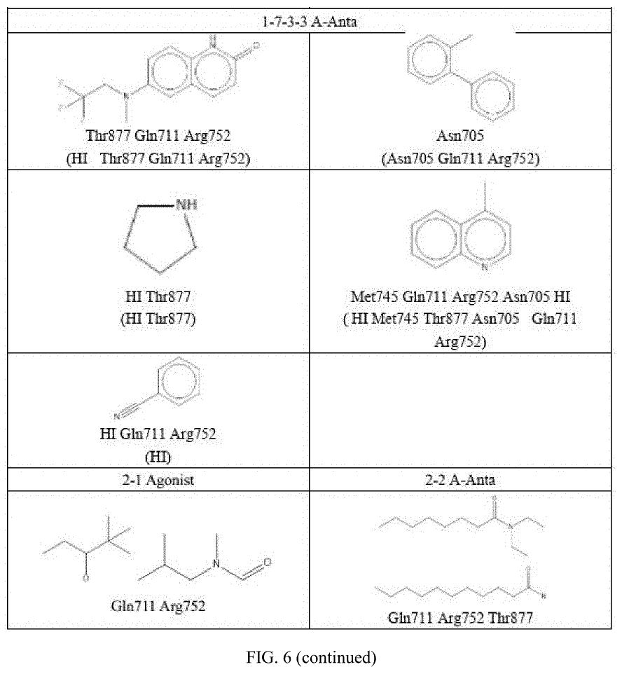

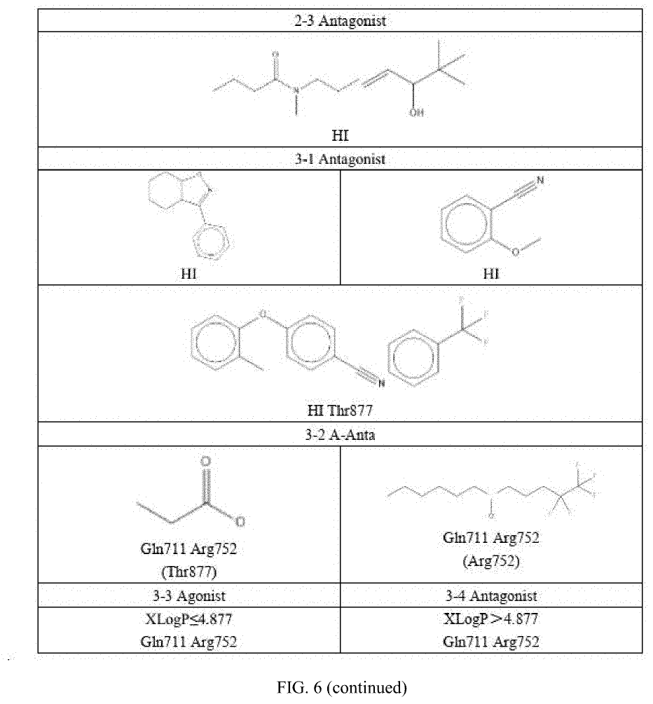

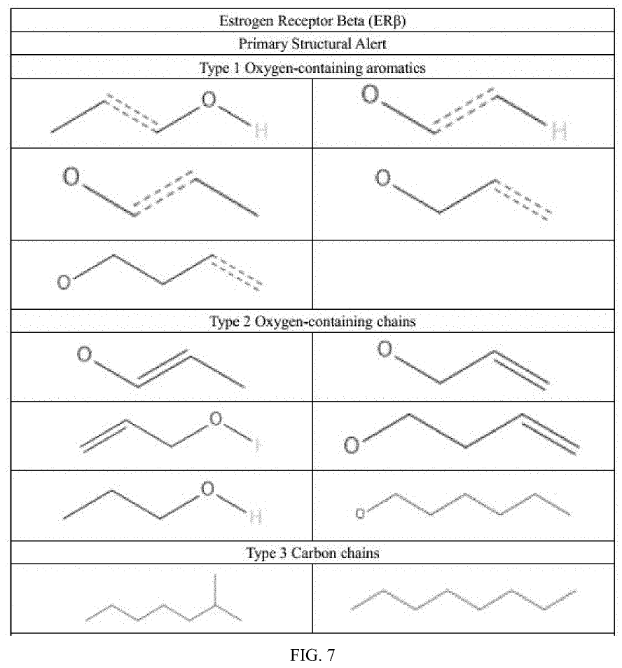

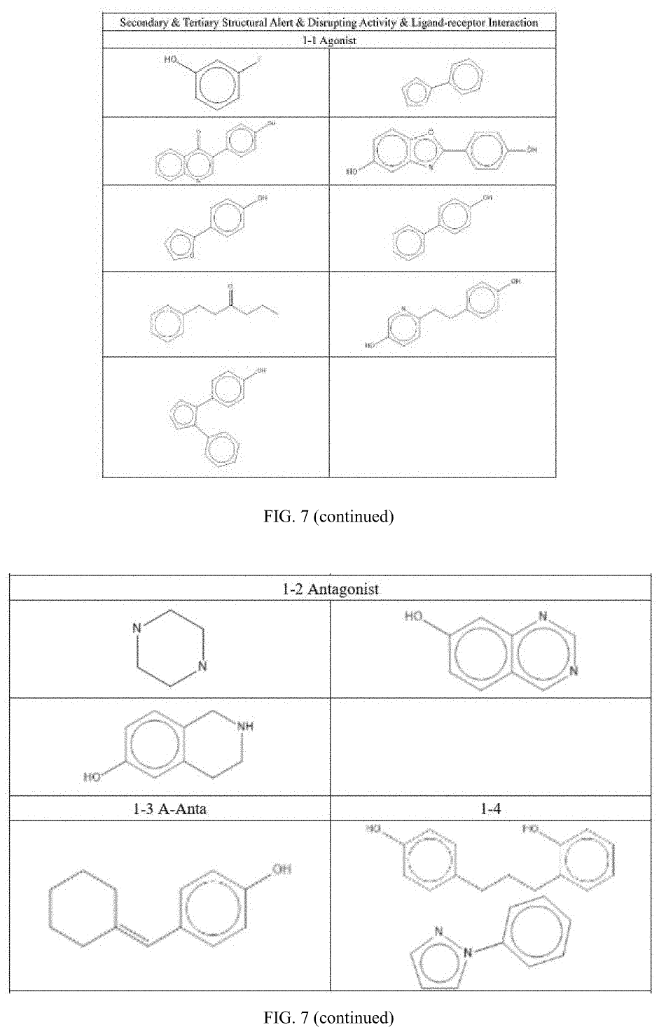

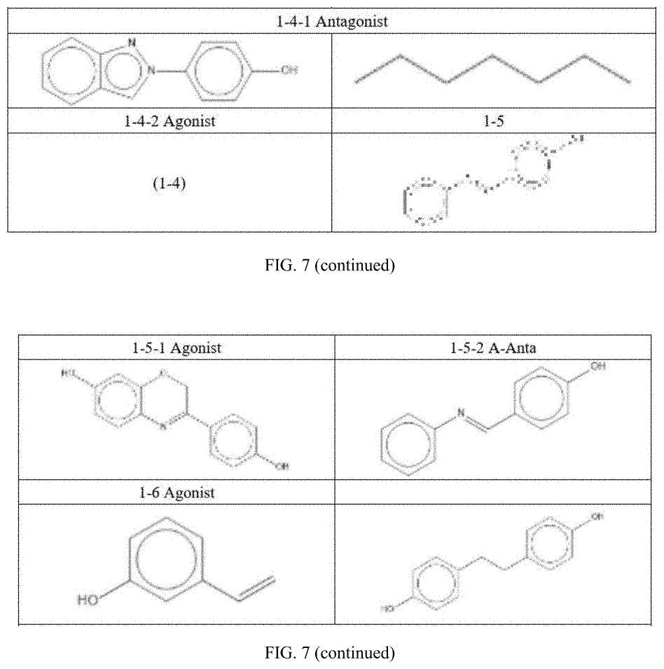

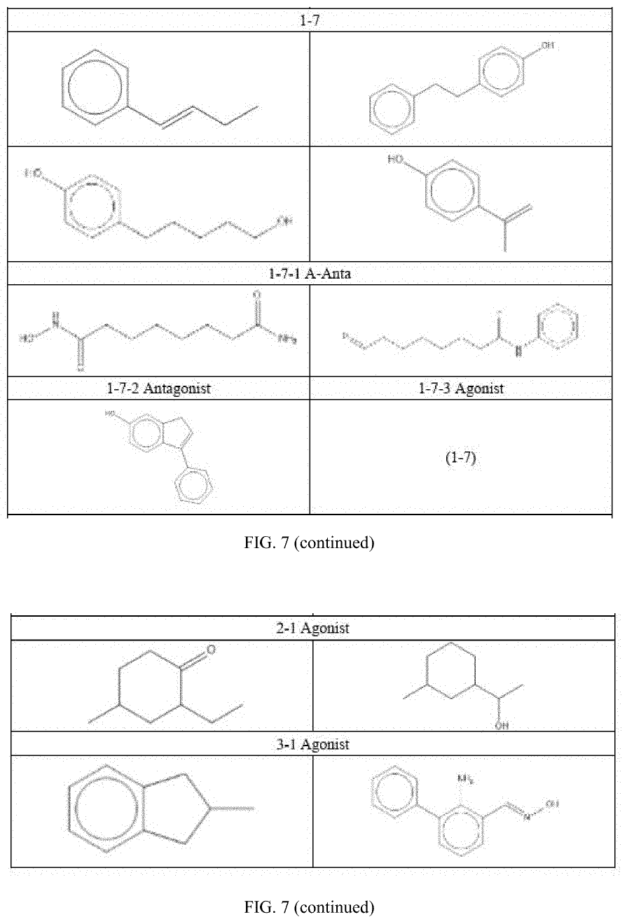

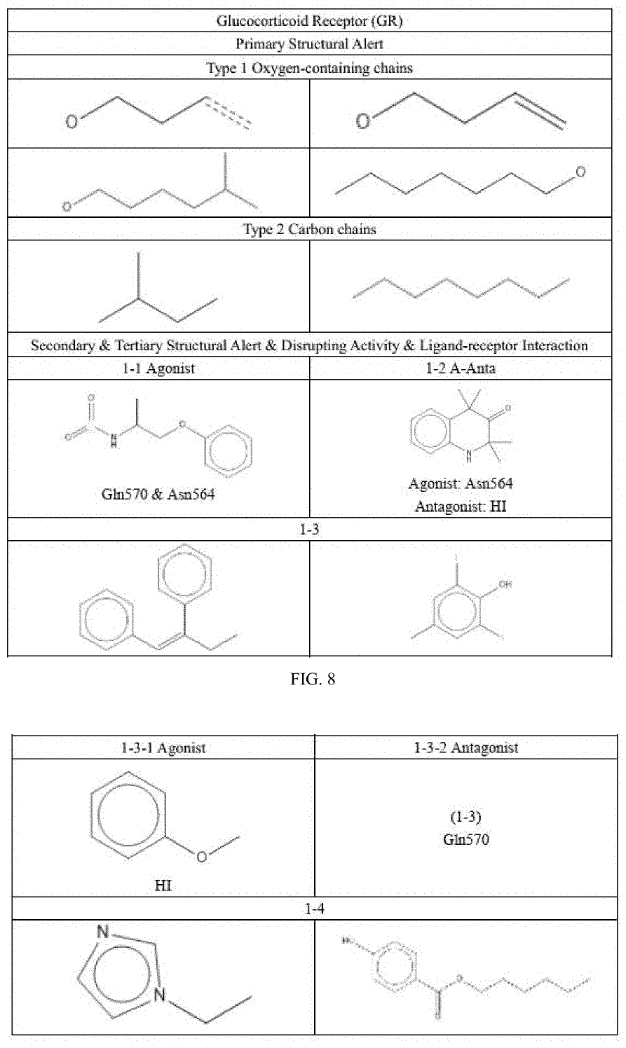

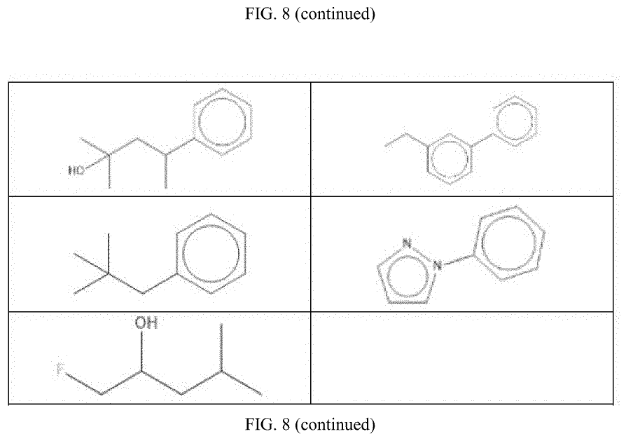

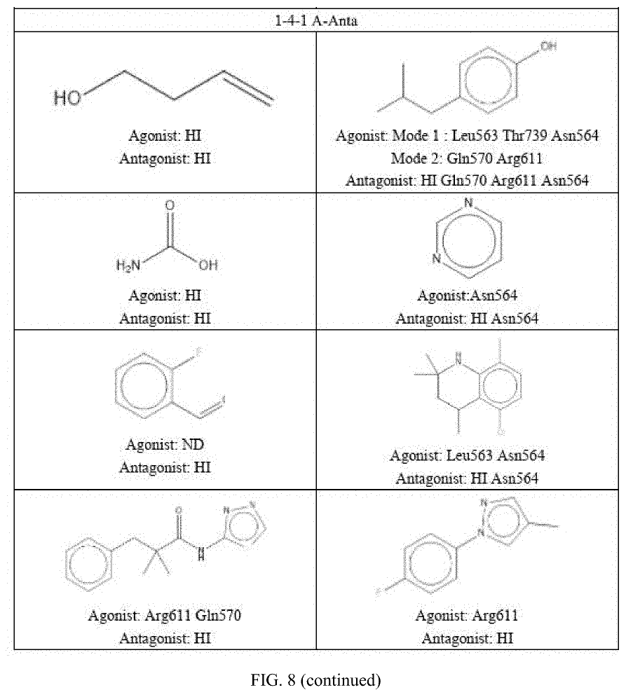

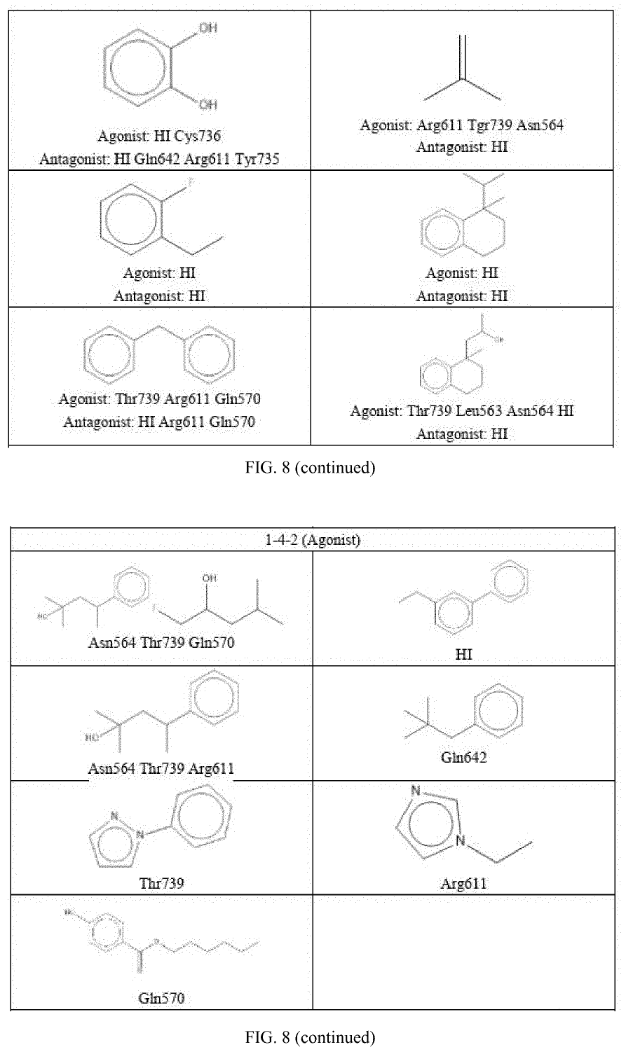

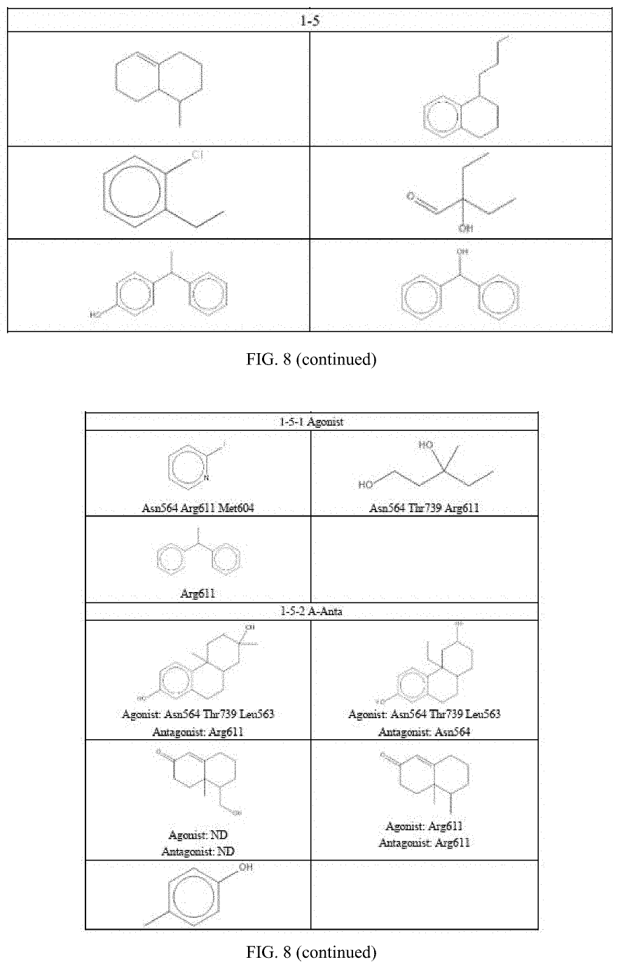

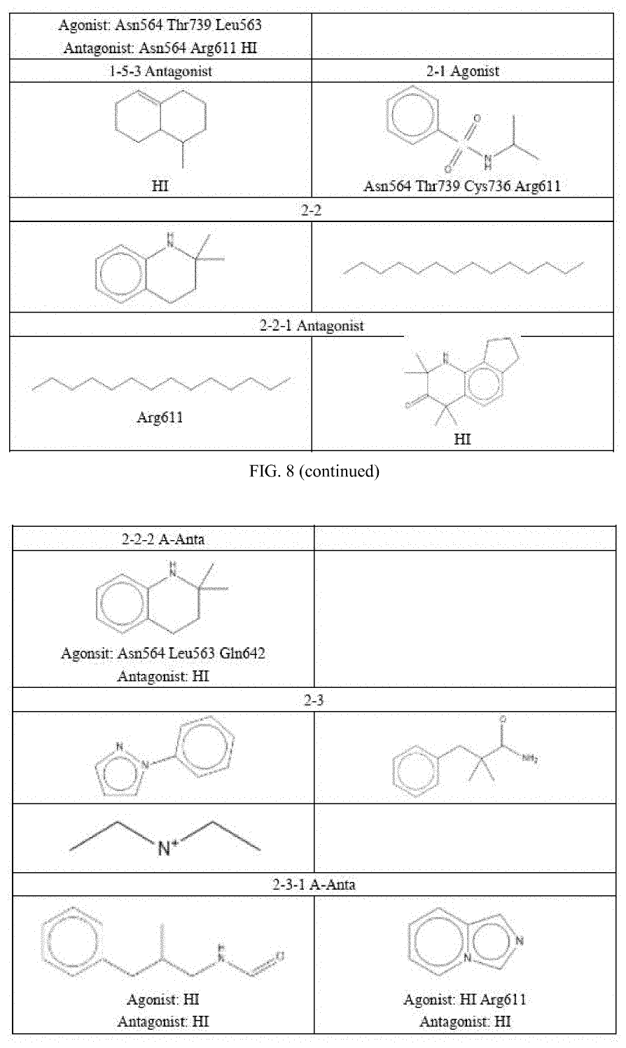

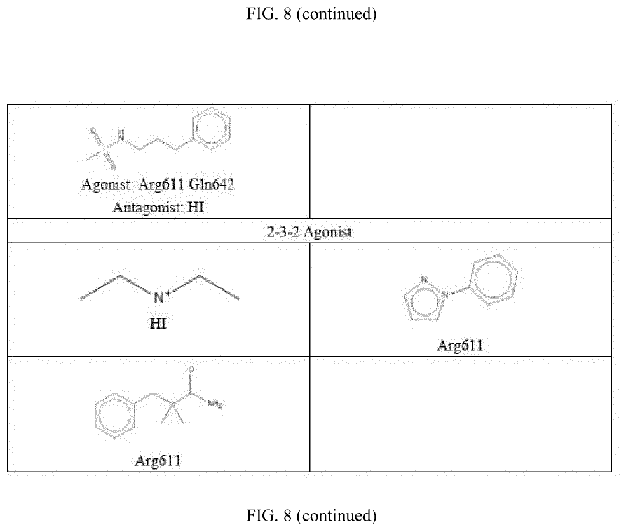

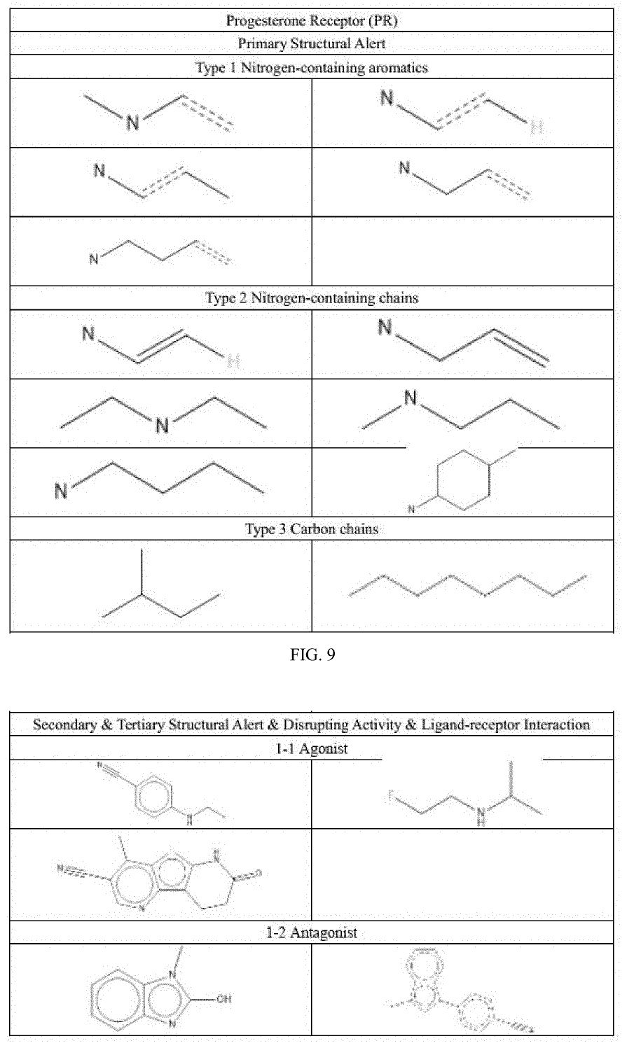

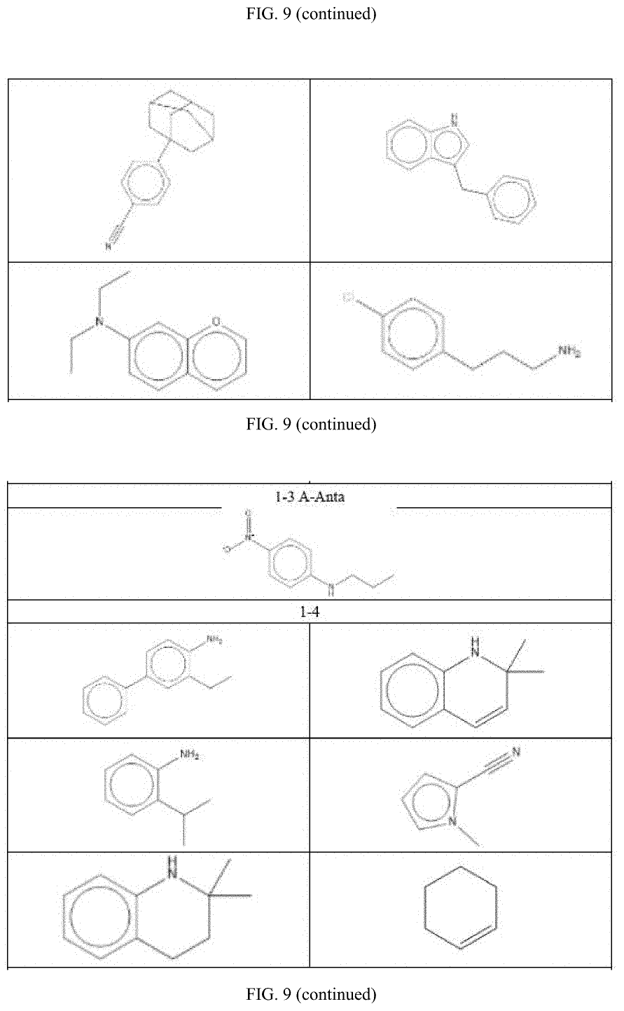

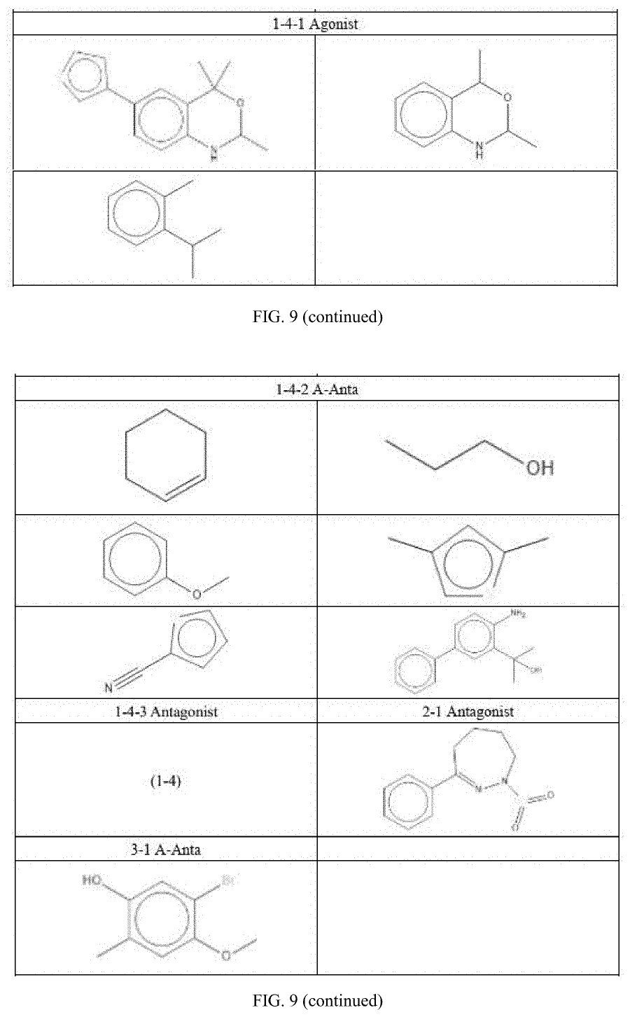

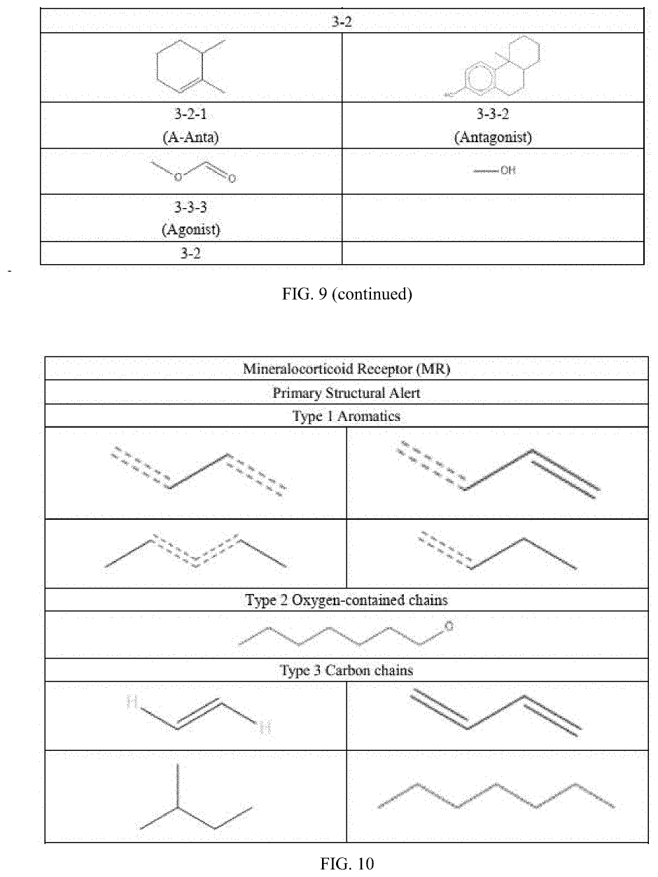

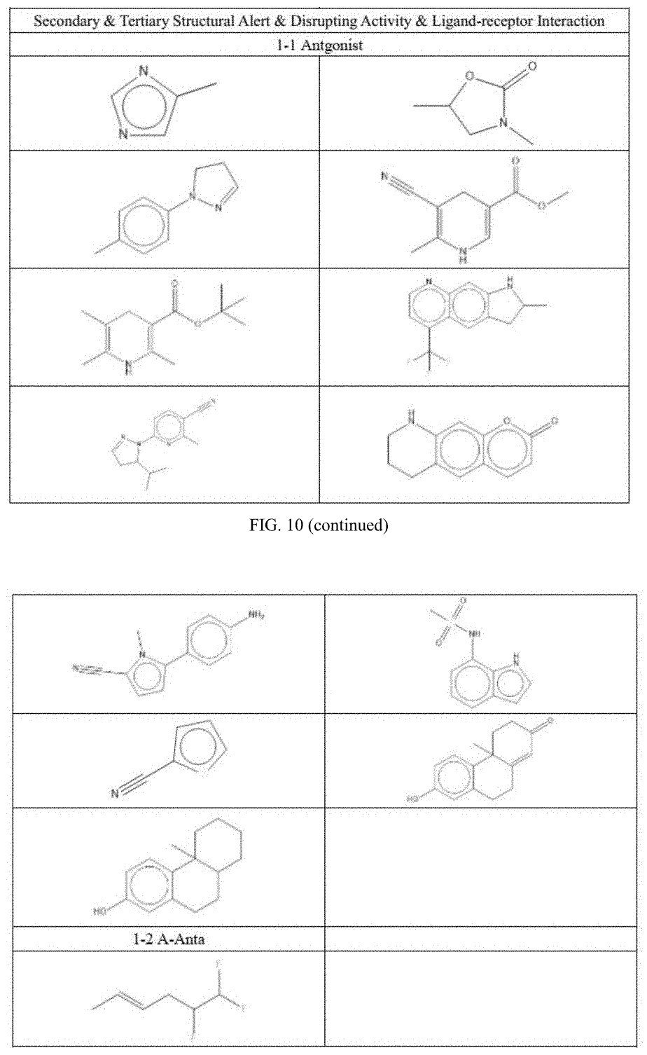

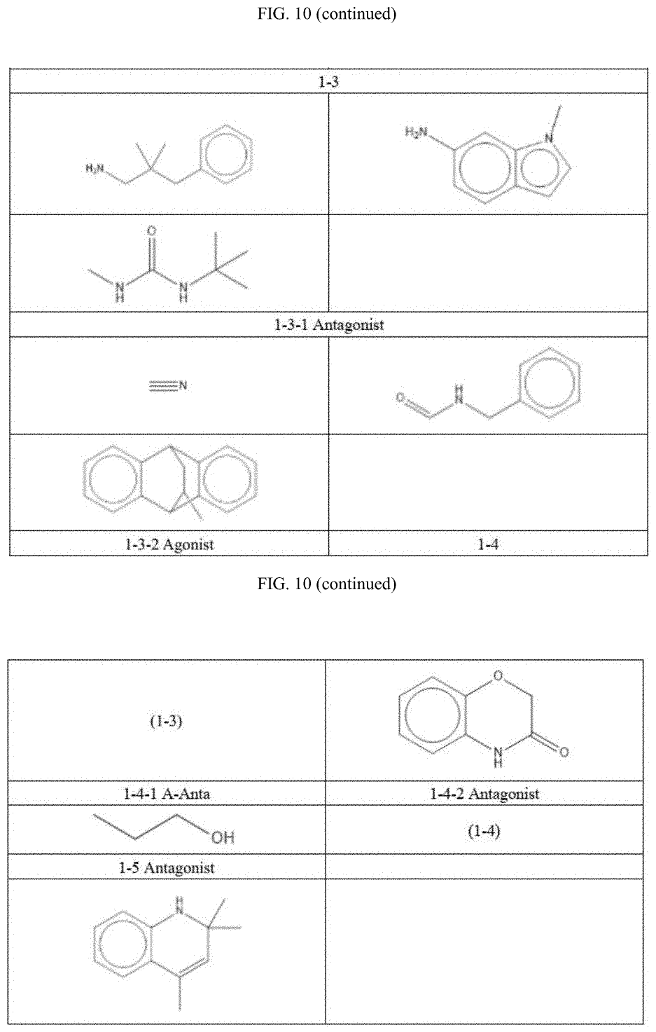

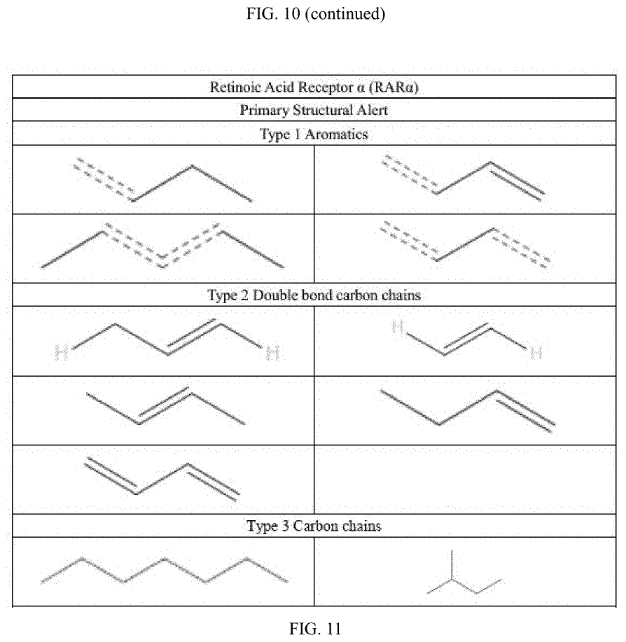

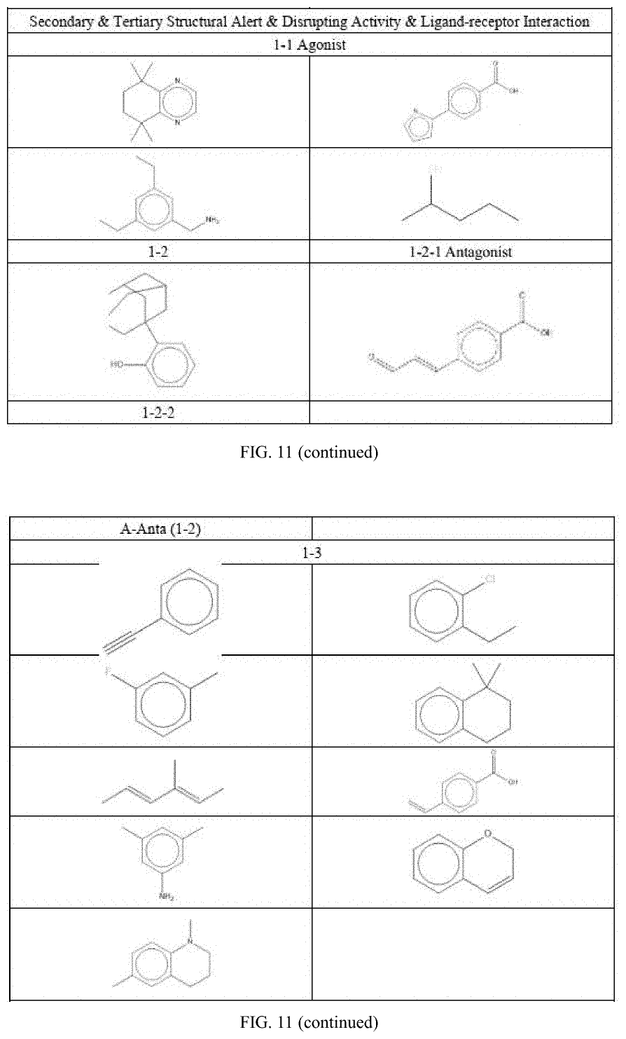

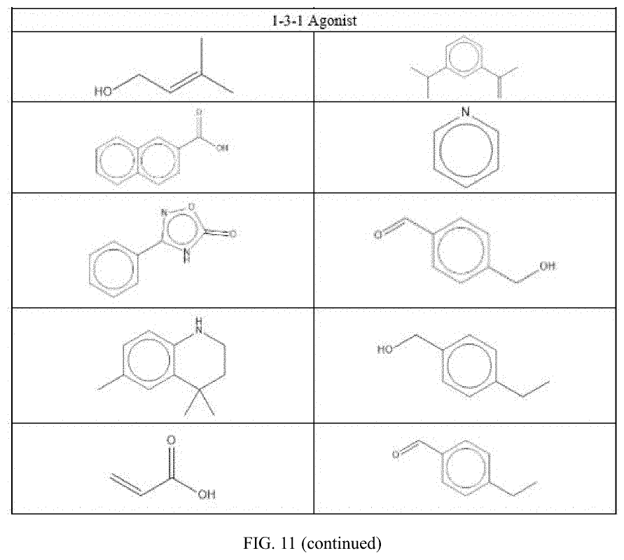

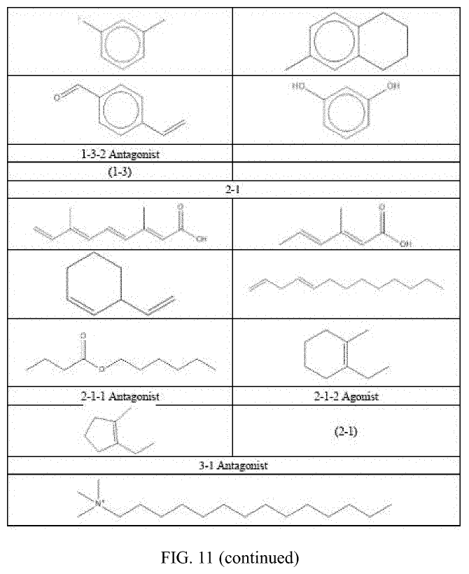

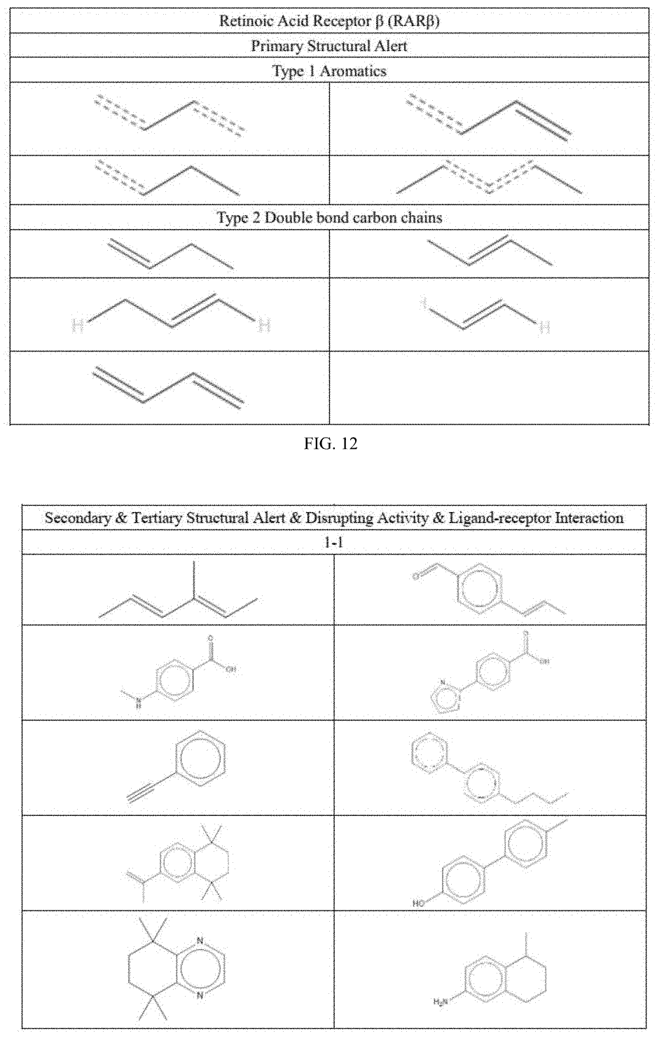

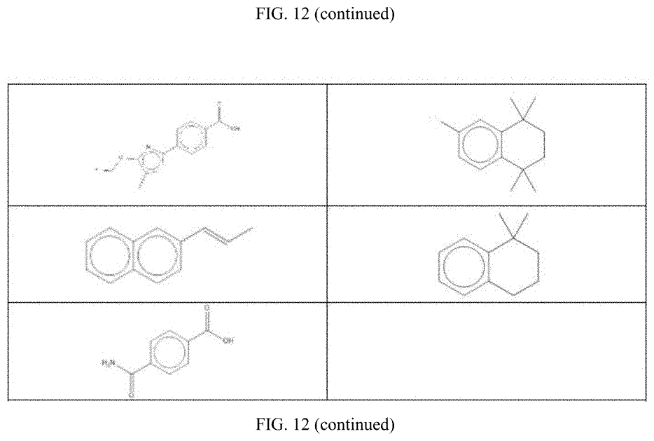

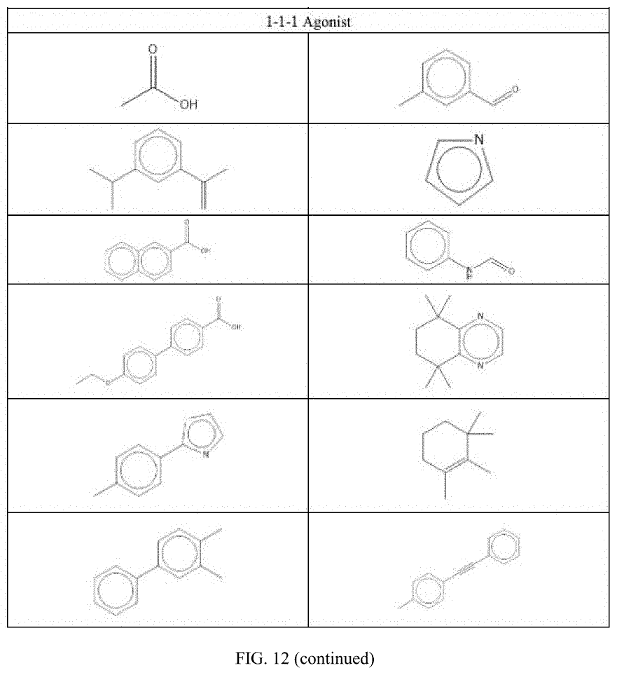

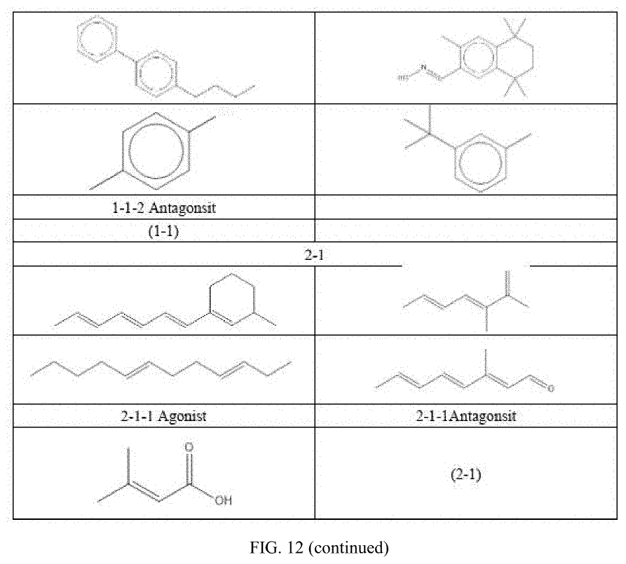

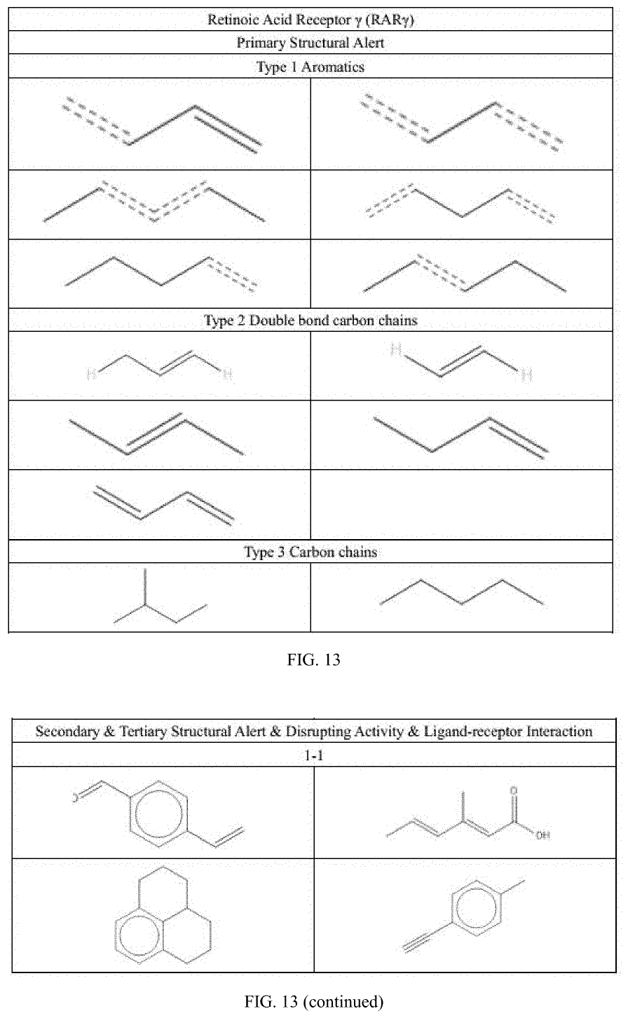

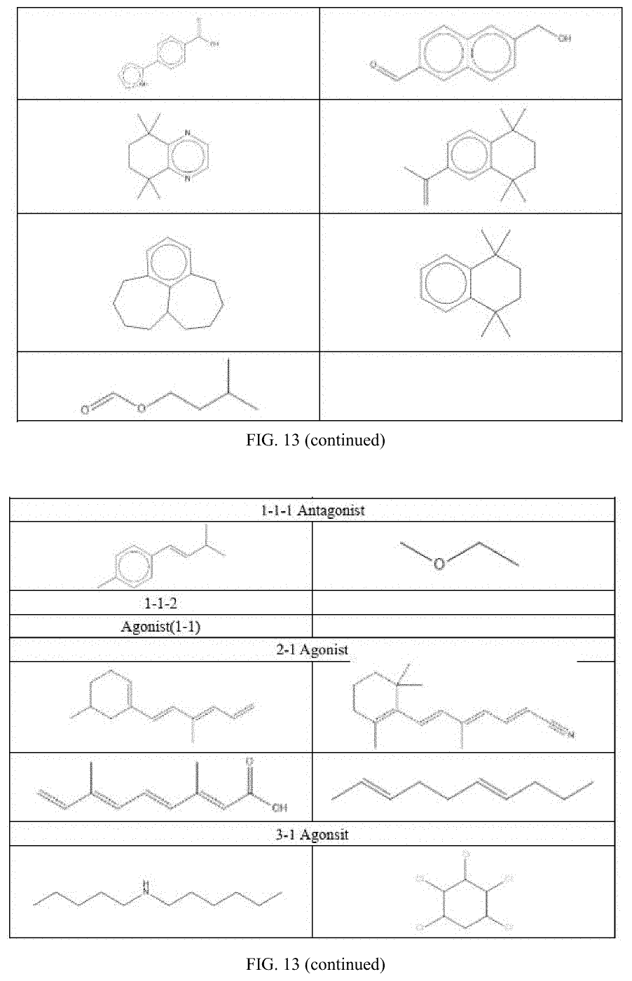

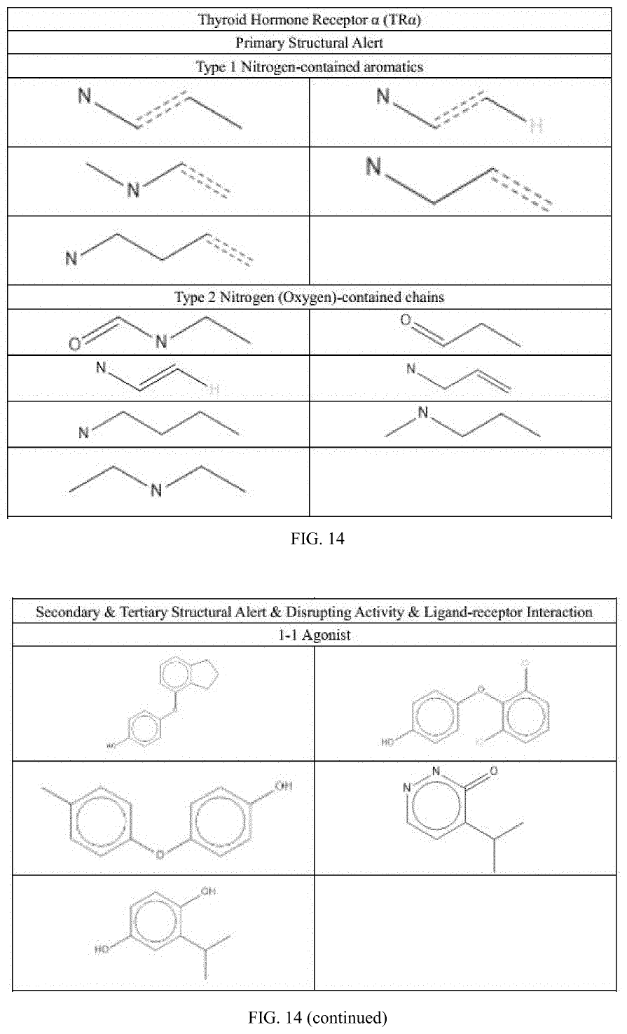

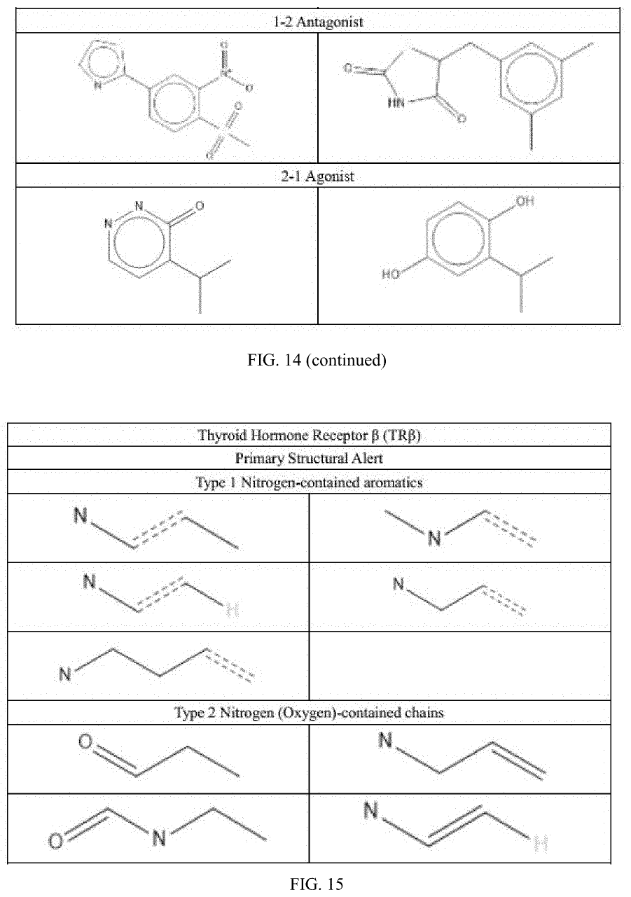

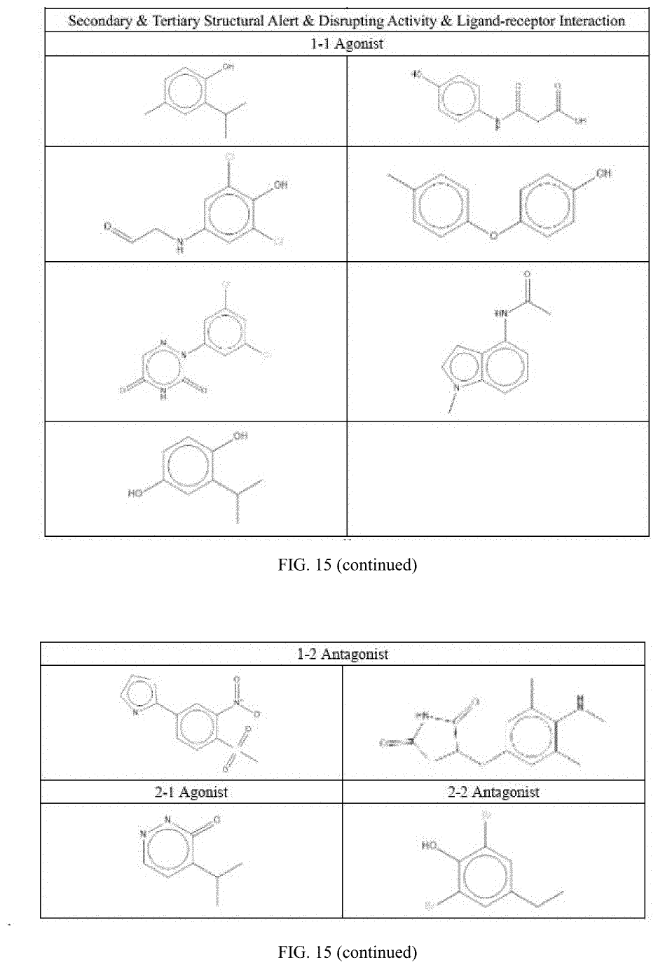

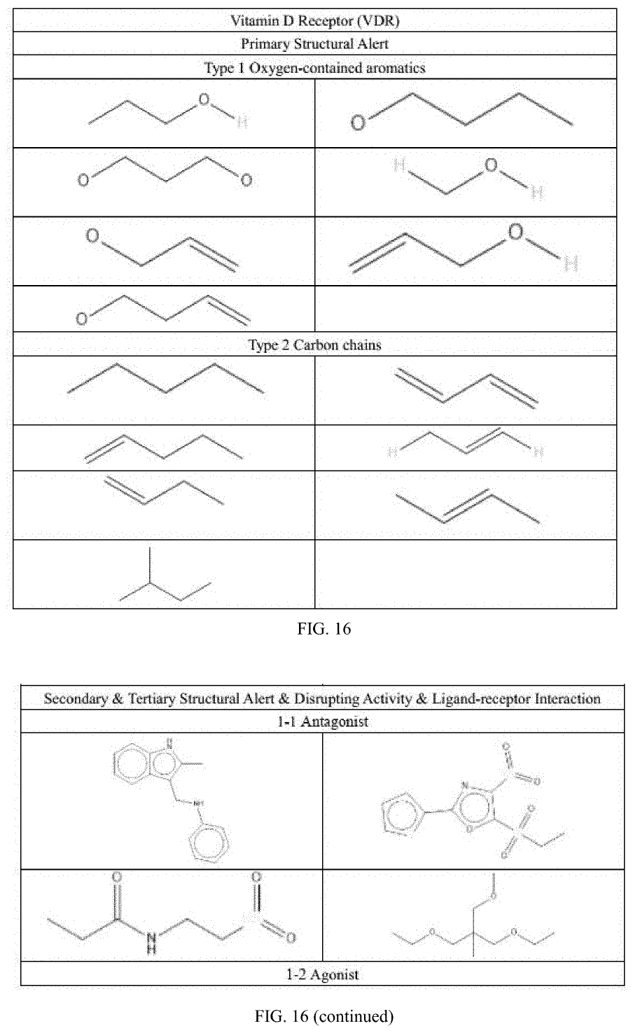

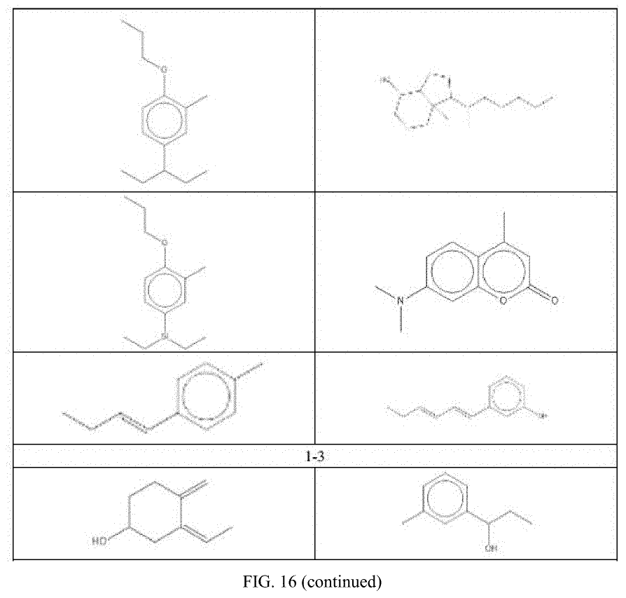

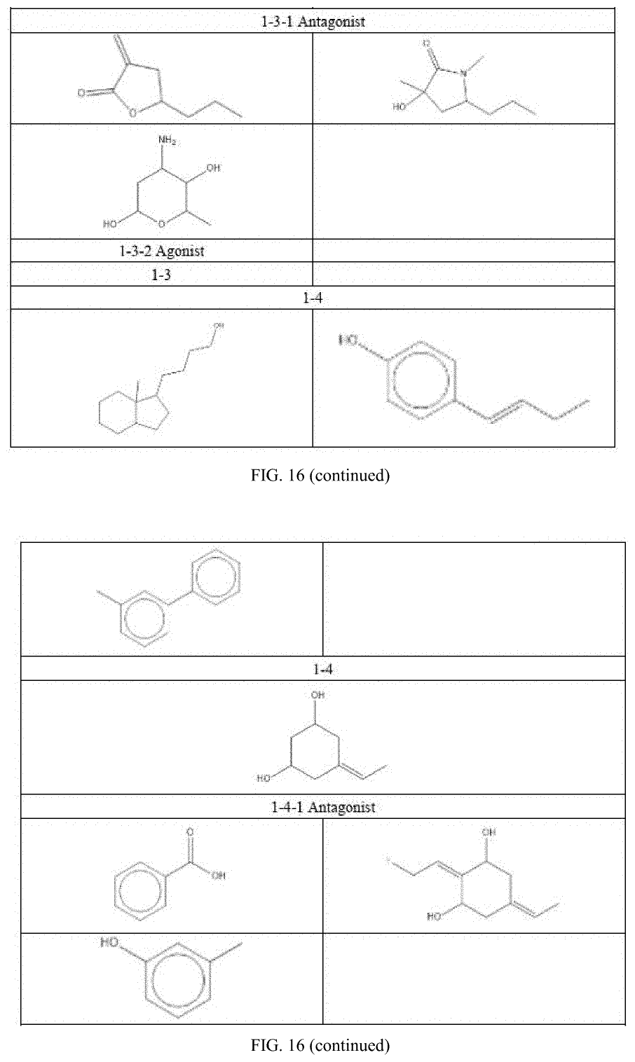

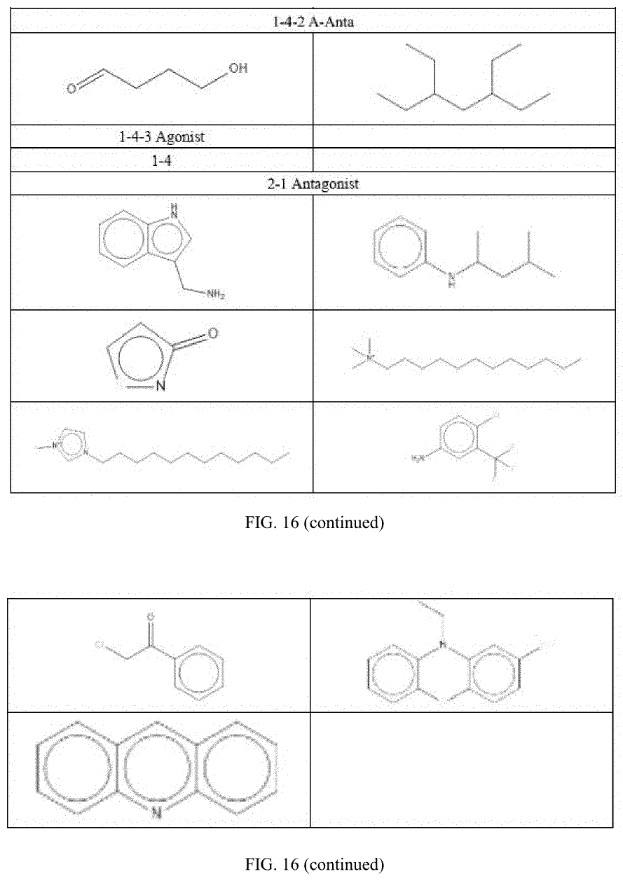

The content of this example is basically the same as that of Example 1, but the difference is that: combining with structural alerts of 12 nuclear receptors shown in FIGS. 5-16, in the model for high-throughput screening of endocrine disruptors and the method for screening the same provided by the present invention, the target nuclear receptor used in this example is human ERα, and the specific steps are as follows:

Acquisition of structural data: according to limitation of the type of the compound in step 1, where the compound in step 1 is an organic compound, the compound Chlorhexidine with the CAS number 55-56-1 is selected as the target compound to predict the disrupting activity. The SMILES number searched from a ChEMBL database and a PubChem database is:

C1C1=CC=C(NC(=N)NC(=N)NCCCCCCNC(=N)NC(=N)NC2=CC=C(COC=C2)C=C1. The SMILES number is imported into ChemBioDraw Ultra 14.0 software for examination. If the structure is correct, then subsequent disrupting activity prediction can be carried out.

Determination of a target nuclear receptor: according to the target nuclear receptor in step 2 is limited to 12 human classical nuclear receptors, the corresponding nuclear receptor high-throughput screening model is selected; and ERα is selected as the target nuclear receptor in this example to predict endocrine disrupting activity.

Matching of primary structural alerts: according to step 3, there are 18 primary structural alerts in ERα (as shown in FIG. 5).

The SMILE number of the target compound Chlorhexidine is imported into PaDEL-descriptor software, Fingerprints is selected in the Descriptors column in General; Remove salt, Detect aromaticity and Standardize nitro groups are selected in Standardize, and a Pubchemfingerpints molecular fingerprint database is checked in Fingerprints to calculate PubChem fingerprints substructure of the compound. Comparing the molecular fingerprint of the obtained target compound with the primary structural alert of the target nuclear receptor, it is found that the compound can meet the seven primary structural alerts in the ERα high-throughput screening model (C:C-C-C,C:C-C:C,C:C-C=C,C-C:C:C-C,C=C-C=C,[#1]-C=C-[#1],C=C-C-C-C).

Matching of secondary structural alerts: according to step 4, 18 primary structural alerts of ERα are divided into four types: structural alerts containing oxygen atoms and aromatic bonds (Type 1), structural alerts containing aromatic bonds (Type 2), carbon chains containing oxygen atoms (Type 3) and any carbon chains without oxygen atoms and aromatic bonds (Type 4). The compounds are divided into three independent large groups according to the descending order of priorities, and there is no intersection between the large groups. Four of the seven primary structural alerts met by the compound belong to Type 2 (C:C-C-C, C:C-C:C, C:C-C=C, C-C:C:C-C), and three belong to Type 4 (C=C-C=C, [#1]-C=C-[#1], C=C-C-C-C). Then the compounds are classified into Type 2 and matching of the secondary structural alerts is performed. There are seven secondary structural alerts in Type 2 of ERα (as shown in FIG. 5). The activity of the target compounds Chlorhexidine is set to 1, a CSV file is formed by using name, structure SMILES number and set activity value of the target compound, the CSV file is imported into SARpy software (http://sarpy.sourceforge.net/), corresponding SMILES column and ACTIVITY attribution are selected in a Get a DATASET module, and then Set numeric threshold is set to 0; LOW<=0<HIGH is selected in Select the desired splitting (threshold belongs to “LOW” or “HIGH” class), Binarize (optional) is checked to set activity >0 to be ACTIVE, set activity <=0 to be INACTIVE, and a Loading button is clicked to import the data set. In Load Ruleset in a Get a RULESET module, SMARTS strings information and Taget (ACTIVE) information of the seven secondary structural alerts are imported in text file format. In a Predict and Validate module, Predict and Validate are clicked, and then Save predictions is clicked; and it is found that the compound meets a secondary structural alert (CNC(=N)NCCC), that is, it is confirmed that the target compound is a disruptor that potentially disrupts ERα nuclear receptor-mediated endocrine regulation system (predicted as ACTIVE).

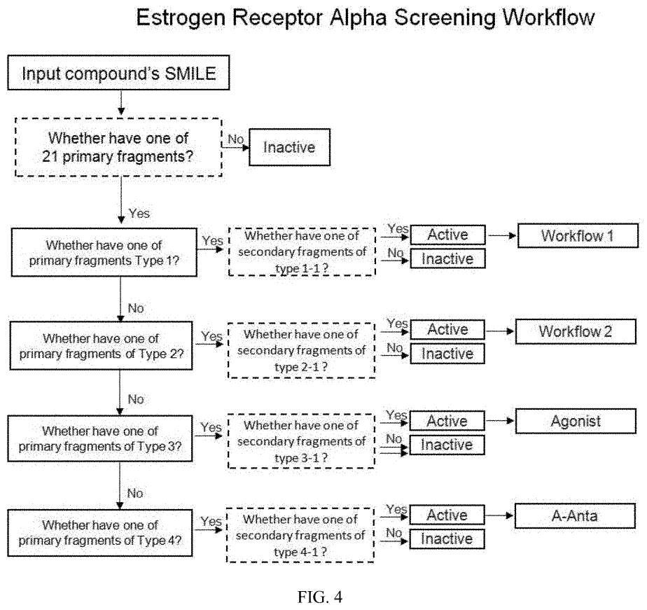

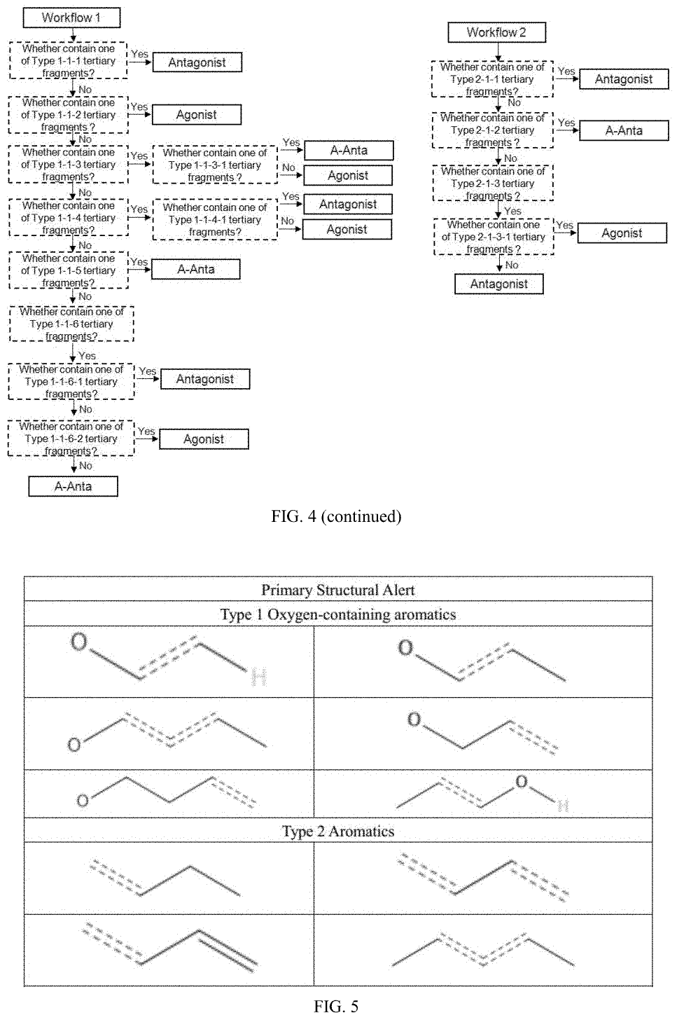

Matching of tertiary structural alerts: according to step 5, there are three types of disruption of agonist, antagonist and A-Anta in Type 2 of ERα. The three types of disruption: agonist, antagonist and A-Anta exist in tertiary structural alerts respectively. The activity of the target compound Chlorhexidine is set to 1, a CSV file is formed by using name, structure SMILES number and set activity value of the target compound, the CSV file is imported into SARpy software (http://sarpy.sourceforge.net/), corresponding SMILES column and ACTIVITY attribution are selected in a Get a DATASET module, and then Set numeric threshold is set to 0; LOW<=0<HIGH is selected in Select the desired splitting (threshold belongs to “LOW” or “HIGH” class), Binarize (optional) is checked to set activity >0 to be ACTIVE, set activity <=0 to be INACTIVE, and a Loading button is clicked to import the data set. In the Load Ruleset in a Get a RULESET module, SMARTS strings information and Taget (Agonist, A-Anta, Antagonist) information of tertiary structural alerts where three types of disruption agonist, A-Anta and antagonist exist respectively are imported in the text file format; in a Predict and Validate module, Predict and Validate are clicked, and then Save predictions is clicked; and it is found that the compound does not have any tertiary structural alert. According to the screening process of ERα disrupting activity (FIG. 4, which shows a prediction process of hierarchical screening of potential active endocrine disruptors based on structural alerts by taking ERα as an example), it is confirmed that the potential disrupting activity of the target compound is antagonistic, that is, there is antagonistic disrupting effect on ERα.

Prediction of ligand-receptor binding mode: according to step 6, based on the primary, secondary and tertiary structural alerts of the target compound Chlorhexidine, it can be determined that the ligand-receptor binding mode existing in the compound and ERα is an antagonistic structure, and the binding mode is mainly His524&Thr347, and the compound has hydrogen bonding with two acute amino acids of His524 and Thr347.

Prediction of binding activity and disrupting activity: according to step 7, compounds with the same structural alert have similar ligand-receptor binding mode, which leads to similar binding activity. When the binding activity is positively correlated with the disrupting activity, it leads to similar disrupting activity, and the binding activity of most compounds is positively correlated with the disrupting activity. Therefore, according to the existing primary, secondary and tertiary structural alerts of the target compound Chlorhexidine, the binding activity intensity of the target compound is predicted to be medium and the disrupting activity is medium.

By constructing a model for high-throughput screening of endocrine disruptors and screening the endocrine disruptors through the model, whether the target compound has disrupting activity and the disrupting activity category for the target nuclear receptor are qualitatively predicted, and competitive binding activity intensity and disrupting activity intensity of the target compound can also be semi-quantitatively predicted based on the similar binding modes of compounds with the same structural alerts.

The present invention has been described in detail above in combination with the specific exemplary embodiments. However, it should be understood that various modifications and variations can be made without departing from the scope of the present invention as defined by the appended claims. The detailed description and accompanying drawings are to be considered as being illustrative only and not restrictive, and if there are any such modifications and variations, they shall fall within the scope of the present invention described herein. In addition, the background is intended to illustrate the research and development status and significance of the technology, and is not intended to limit the present invention or the present application and application fields of the present invention.

What is claimed is:

1. A model for high-throughput screening of endocrine disruptors, wherein
primary structural alerts, secondary structural alerts and tertiary structural alerts of compounds are extracted according to a nuclear receptor, and wherein based on a PubChem fingerprint molecular fingerprint database, the primary structural alerts of the compounds are extracted through substructure frequency analysis and substructure percentage analysis; the secondary structural alerts are extracted from compounds meeting the primary structural alerts by using SARpy software; the tertiary structural alerts are extracted from compounds meeting both the primary structural alerts and the secondary structural alerts by using the SARpy software; and
the primary structural alerts, the secondary structural alerts and the tertiary structural alerts described above jointly form a nuclear receptor high-throughput screening model.

2. The model for high-throughput screening of endocrine disruptors according to claim 1, wherein the nuclear receptor is any one of androgen receptor (AR), estrogen receptor α (ERα), estrogen receptor β (ERβ), glucocorticoid receptor (GR), mineralocorticoid receptor (MR), progesterone receptor (PR), retinoic acid receptor α (RARα), retinoic acid receptor β (RARβ), retinoic acid receptor γ (RARγ), thyroid hormone receptor α (TRα), thyroid hormone receptor β (TRβ) and vitamin D receptor (VDR).

3. The model for high-throughput screening of endocrine disruptors according to claim 1, wherein
the substructure frequency analysis is performed by using formula
$$Frequency of a fragment = \frac{( {N}_{fragment}^{A} \times {N}_{total} )}{( {N}_{fragment \_ total} \times {N}_{A} )} ,$$
the substructure percentage analysis is performed by using formula
$$Percentage of a fragment = \frac{{N}_{fragment}^{A}}{{N}_{A}} or \frac{{N}_{fragment}^{I}}{{N}_{I}} ,$$
wherein the above-mentioned
Frequency of a fragment denotes substructure frequency;
Percentage of a fragment denotes substructure percentage;
N_{fragment}^{A} is the number of active compounds containing the structural fragment;
Nₜₒₜₐₗ is the total number of compounds in a whole data set;
N_{fragment_total} is the total number of compounds containing the structural fragment;
N_{A} is the total number of active compounds in the data set;
N_{fragment}^{I} is the total number of inactive compounds containing the structural fragment; and
N_{I} is the total number of inactive compounds in the data set.

4. A method for high-throughput screening of endocrine disruptors, wherein the method uses the model for high-throughput screening of endocrine disruptors according to claim 1, and comprises the following steps:
step 1: acquisition of structural data
selecting chemical structures of target compounds without relevant activity data from a public database, and denoting the chemical structures by SMILES numbers;
step 2: determination of a target nuclear receptor
determining a target nuclear receptor based on the disrupting activity of the target compounds to be predicted;
step 3: matching of primary structural alerts
selecting a target nuclear receptor high-throughput screening model based on the determined target nuclear receptor, calculating molecular fingerprints of the target compounds by using a PubChem fingerprints molecular fingerprint database of PaDEL-Descriptor software, and matching a calculated result with primary structural alerts of the target nuclear receptor high-throughput screening model;
step 4: matching of secondary structural alerts
grouping target compounds meeting the primary structural alerts based on the primary structural alerts, and matching the target compounds with secondary structural alerts of the target nuclear receptor high-throughput screening model by using SARpy software; and
step 5: matching of tertiary structural alerts
grouping target compounds meeting both the primary structural alerts and the secondary structural alerts based on the secondary structural alerts, and matching with tertiary structural alerts of the target nuclear receptor high-throughput screening model by using the SARpy software.

5. The method for high-throughput screening of endocrine disruptors according to claim 4, wherein a method for grouping the target compounds in step 4 comprises: grouping the primary structural alerts of the target nuclear receptor high-throughput screening model first, and taking the primary structural alerts containing polar atoms and aromatic ring bonds as Type 1; taking the primary structural alerts containing aromatic bonds as Type 2; taking the primary structural alerts of carbon chains containing oxygen atoms as Type 3; finally taking the primary structural alerts of any carbon chains without oxygen atoms and aromatic bonds as Type 4; wherein priorities of Type 1, Type 2, Type 3 and Type 4 are successively decreased, matching the primary structural alerts of the target compounds with the primary structural alerts in each group, and then classifying the target compounds from the group with successful matching to the group with the highest priority.

6. The method for high-throughput screening of endocrine disruptors according to claim 4, wherein a method for grouping the target compounds in step 5 comprises: grouping the secondary structural alerts of the target nuclear receptor high-throughput screening model first, and taking the secondary structural alerts existing only in antagonistic compounds as Type 1-1, taking the secondary structural alerts existing only in agonistic compounds as Type 1-2, and finally taking the secondary structural alerts existing in agonistic-antagonistic (A-Anta) compounds as Type 1-3; wherein priorities of Type 1-1, Type 1-2 and Type 1-3 are successively decreased, matching the secondary structural alerts of the target compounds with the secondary structural alerts in each group, and then classifying the target compounds from the group with successful matching to the group with the highest priority.

7. The method for high-throughput screening of endocrine disruptors according to claim 4, further comprising:
step 6: analysis of ligand-receptor binding mode
selecting target compounds meeting the primary structural alerts, the secondary structural alerts and the tertiary structural alerts through the target nuclear receptor high-throughput screening model, selecting a pattern protein receptor crystal from RCSB PDB Database based on the disrupting activity of the target compounds, performing molecular docking by using Autodock vina software, and performing ligand-receptor binding mode analysis by using Ligplus software;
step 7: semi-quantitative prediction of binding activity and disrupting activity
wherein target compounds with the same structural alert have similar ligand-receptor binding mode and similar binding activity; when the binding activity is positively correlated with the disrupting activity, grouping the target compounds based on the primary structural alerts, the secondary structural alerts and the tertiary structural alerts of the target compounds, and semi-quantitatively predicting the binding activity and the disrupting activity of the target compounds.

8. The method for high-throughput screening of endocrine disruptors according to claim 7, wherein
the binding activity and the disrupting activity are standardized and classified in intensity by using formula
$$Activity value = - {log}_{10} \frac{{K}_{i} , {K}_{d} , {AC}_{50} , {IC}_{50} , {EC}_{50} ( uM )}{1000000} ;$$
wherein Activity value denotes a value of activity intensity, Kᵢ denotes an inhibition constant, K_{d} denotes a dissociation constant, AC₅₀ denotes a median activity concentration, IC₅₀ denotes a median inhibitory concentration, EC₅₀ denotes a median effect concentration, uM denotes a micromolar weight, Activity value ≥7 denotes a strong activity intensity, 7>Activity value ≥6 denotes a medium activity intensity, and Activity value <6 denotes a weak activity intensity.

9. The method for high-throughput screening of endocrine disruptors according to claim 7, wherein the target nuclear receptor high-throughput screening model is any one of an AR high-throughput screening model, an ERα high-throughput screening model and a GR high-throughput screening model.

10. The method for high-throughput screening of endocrine disruptors according to claim 9, wherein the target compounds are organic compounds.
